(19) United States
(12) Patent Application Publication
(10) Pub. No.: US 2026/0133199 A1
(43) Pub. Date: May 14, 2026

(54) Biomarkers for the early detection of breast cancer

(71) Applicants: ARIZONA BOARD OF REGENTS, a body corporate acting for and on behalf of Arizona State University, Scottsdale, AZ (US); DANA-FARBER CANCER INSTITUTE, INC., Boston, MA (US); President and Fellows of Harvard College, Cambridge, MA (US)

(72) Inventors: Joshua LABAER, Chandler, AZ (US); Karen Sue ANDERSON, Chestnut Hill, MA (US); Garrick WALLSTROM, Mesa, AZ (US); Sahar SIBANI, Revere, MA (US); Niroshan RAMACHANDRAN, San Marcos, CA (US)

(21) Appl. No.: 19/440,130

(22) Filed: Jan. 5, 2026

Related U.S. Application Data
(62) Division of application No. 18/173,892, filed on Feb. 24, 2023, now Pat. No. 12,540,940.
Application No. 18/173,892 is a division of application No. 17/015,702, filed on Sep. 9, 2020, now Pat. No. 11,624,747.
Application No. 17/015,702 is a division of application No. 15/818,975, filed on Nov. 21, 2017, now Pat. No. 10,802,026.
Application No. 15/818,975 is a continuation of application No. 13/809,695, now Pat. No. 9,857,374.
(60) Provisional application No. 61/373,359, filed on Aug. 13, 2010.

(51) Int. Cl. G01N 33/57515; C07K 14/47; C07K 17/00; C12Q 1/6886; C40B 40/10

(52) U.S. Cl. CPC G01N 33/57515; C07K 14/47; C07K 17/00; C12Q 1/6886; C40B 40/10; C12Q 2600/158; C12Y 301/27004

(57) ABSTRACT

The present invention provides reagents and methods for breast cancer detection.

### CROSS-REFERENCE

This application is a divisional of U.S. application Ser. No. 18/173,892, filed Feb. 24, 2023; which is a divisional of U.S. application Ser. No. 17/015,702, filed Sep. 20, 2020, which issued as a U.S. Pat. No. 11,624,747, issued Apr. 11, 2023; which is a divisional of U.S. application Ser. No. 15/818,975, filed Nov. 21, 2017, which issued as a U.S. Pat. No. 10,802,026, issued Oct. 13, 2020; which is a continuation of U.S. application Ser. No. 13/809,695, filed Feb. 27, 2013, which issued as a U.S. Pat. No. 9,857,374, issued Jan. 2, 2018; which is a national stage application under 35 U.S.C. § 371 of International Application No. PCT/US2011/047741 filed Aug. 15, 2011; which claims priority to U.S. Provisional Patent Application Ser. No. 61/373,359 filed Aug. 13, 2010, incorporated by reference herein in their entireties.

### STATEMENT OF U.S. GOVERNMENT INTEREST

This invention was made with government support under P50 CA089393 and U01 CA117374 awarded by the National Institutes of Health. The government has certain rights in the invention.

### SEQUENCE LISTING STATEMENT

The sequence listing is filed in this application in electronic format only and is incorporated by reference herein. The sequence listing text file “10-294-PCT.xml” was created on Feb. 23, 2023, and is 95,937 bytes in size.

### BACKGROUND

Despite recent advances in early detection and treatment, breast cancer remains a common and significant health problem in the United States. Women diagnosed with stage II and III breast cancer have a high-risk for distant recurrence and up to half of these women will develop metastatic disease, which remains incurable with current therapy. In this setting, there is intense effort in the search for biomarkers that can detect early disease, and to monitor for disease progression and recurrence. With the advent of molecularly-targeted therapeutics, biomarkers that are associated with biological subtypes of cancer may be useful for predicting responses to therapeutic interventions.

Proteomics-based approaches to distinguish cancer-bearing patient sera from healthy control sera have been challenged by the difficulty in identifying small quantities of protein fragments within complex protein mixtures, protein instability, and natural variations in protein content within patient populations. Autoantibodies (AAb) to tumor antigens have advantages over other serum proteins as potential cancer biomarkers as they are stable, highly specific, easily purified from serum, and are readily detected with well-validated secondary reagents. Although they have high specificities to distinguish cancer from control sera, most tumor AAb demonstrate poor sensitivities. Testing multiple antigens in parallel may serve to increase the predictive value of tumor-specific antibodies for use as immunodiagnostics.

Protein microarrays offer an emerging platform to present tumor antigens to screen for immune responses. In comparison to traditional ELISAs, protein microarrays are capable of presenting and assessing hundreds of tumor antigens simultaneously. The responses are rapidly identified because the address of each protein is known in advance and there are no representation issues; all proteins, even rare ones, are represented equally (usually in duplicate). The proteins are arrayed on a single microscope slide requiring only a few microliters of serum per assay. Known tumor antigens as well as predicted tumor antigens can be included to generate a comprehensive protein tumor antigen array. Despite some early demonstrations of feasibility, protein microarrays are not yet widely used, due to the labor and technical issues associated with production, purification, and quality control of proteins for spotting on the array, as well as difficulties with downstream validation assays of target AAb.

### SUMMARY OF THE INVENTION

In a first aspect, the present invention provides polypeptide probe sets comprising:
at least 2 different isolated polypeptides selected from the group consisting of ATP6AP1 (SEQ ID NO: 13), PDCD6IP (SEQ ID NO: 21), DBT (SEQ ID NO: 25), CSNKIE (SEQ ID NO: 9), FRS3 (SEQ ID NO: 3), RAC3 (SEQ ID NO: 15), HOXD1 (SEQ ID NO: 7), SF3A1 (SEQ ID NO: 1), CTBP1 (SEQ ID NO: 29), C15orf48 (SEQ ID NO: 35), MYOZ2 (SEQ ID NO: 33), EIF3E (SEQ ID NO: 39), BAT4 (SEQ ID NO: 5), ATF3 (SEQ ID NO: 19), BMX (SEQ ID NO: 45), RAB5A (SEQ ID NO: 23), UBAP1 (SEQ ID NO: 47), SOX2 (SEQ ID NO: 31), GPR157 (SEQ ID NO: 43), BDNF (SEQ ID NO: 17), ZMYM6 (SEQ ID NO: 41), SLC33A1 (SEQ ID NO: 11), TRIM32 (SEQ ID NO: 37), ALG10 (SEQ ID NO: 27), TFCP2 (SEQ ID NO: 49), SERPINH1 (SEQ ID NO: 51), SELL (SEQ ID NO: 55), ZNF510 (SEQ ID NO: 53), or antigenic fragments thereof, attached to the support.

In a second aspect, the present invention provides polynucleotide arrays comprising:
(a) a support; and
(b) at least 2 different isolated nucleic acids encoding polypeptides selected from the group consisting of ATP6AP1 (SEQ ID NO: 14), PDCD6IP (SEQ ID NO: 22), DBT (SEQ ID NO: 26), CSNKIE (SEQ ID NO: 10), FRS3 (SEQ ID NO: 4), RAC3 (SEQ ID NO: 16), HOXD1 (SEQ ID NO: 8), SF3A1 (SEQ ID NO: 2), CTBP1 (SEQ ID NO: 30), C15orf48 (SEQ ID NO: 36), MYOZ2 (SEQ ID NO: 34), EIF3E (SEQ ID NO: 40), BAT4 (SEQ ID NO: 6), ATF3 (SEQ ID NO: 20), BMX (SEQ ID NO: 46), RAB5A (SEQ ID NO: 24), UBAP1 (SEQ ID NO: 48), SOX2 (SEQ ID NO: 32), GPR157 (SEQ ID NO: 44), BDNF (SEQ ID NO: 18), ZMYM6 (SEQ ID NO: 42), SLC33A1 (SEQ ID NO: 12), TRIM32 (SEQ ID NO: 38), ALG10 (SEQ ID NO: 28), TFCP2 (SEQ ID NO: 50), SERPINH1 (SEQ ID NO: 52), SELL (SEQ ID NO: 56), ZNF510 (SEQ ID NO: 54), or antigenic fragments thereof, attached to the support.

In a third aspect, the present invention provides methods for detecting breast cancer, comprising;
(a) contacting a suitable bodily fluid sample obtained from a subject at risk of breast cancer with one or more isolated polypeptides selected from the group consisting of ATP6AP1 (SEQ ID NO: 13), PDCD6IP (SEQ ID NO: 21), DBT (SEQ ID NO: 25), CSNKIE (SEQ ID NO: 9), FRS3 (SEQ ID NO: 3), RAC3 (SEQ ID NO: 15), HOXD1 (SEQ ID NO: 7), SF3A1 (SEQ ID NO: 1), CTBP1 (SEQ ID NO: 29), C15orf48 (SEQ ID NO: 35), MYOZ2 (SEQ ID NO: 33), EIF3E (SEQ ID NO: 39), BAT4 (SEQ ID NO: 5), ATF3 (SEQ ID NO: 19), BMX (SEQ ID NO: 45), RAB5A (SEQ ID NO: 23), UBAP1 (SEQ ID NO: 47), SOX2 (SEQ ID NO: 31), GPR157 (SEQ ID NO: 43), BDNF (SEQ ID NO: 17), ZMYM6 (SEQ ID NO: 41), SLC33A1 (SEQ ID NO: 11), TRIM32 (SEQ ID NO: 37), ALG10 (SEQ ID NO: 27), TFCP2 (SEQ ID NO: 49), SERPINH1 (SEQ ID NO: 51), SELL (SEQ ID NO: 55), ZNF510 (SEQ ID NO: 53), or antigenic fragments thereof; wherein the contacting occurs under conditions suitable for selective binding of antibodies in the bodily fluid sample to the one or more polypeptides; and
(b) detecting presence of antibodies to the polypeptides in the bodily fluid sample;
wherein the presence of antibodies to the one or more polypeptides indicates a likelihood of breast cancer in the subject.

### DETAILED DESCRIPTION OF THE INVENTION

In a first aspect, the present invention provides polypeptide probe sets comprising:
(a) at least 2 different isolated polypeptides selected from the group consisting of ATP6AP1 (SEQ ID NO: 13), PDCD6IP (SEQ ID NO: 21), DBT (SEQ ID NO: 25), CSNKIE (SEQ ID NO: 9), FRS3 (SEQ ID NO: 3), RAC3 (SEQ ID NO: 15), HOXD1 (SEQ ID NO: 7), SF3A1 (SEQ ID NO: 1), CTBP1 (SEQ ID NO: 29), C15orf48 (SEQ ID NO: 35), MYOZ2 (SEQ ID NO: 33), EIF3E (SEQ ID NO: 39), BAT4 (SEQ ID NO: 5), ATF3 (SEQ ID NO: 19), BMX (SEQ ID NO: 45), RAB5A (SEQ ID NO: 23), UBAP1 (SEQ ID NO: 47), SOX2 (SEQ ID NO: 31), GPR157 (SEQ ID NO: 43), BDNF (SEQ ID NO: 17), ZMYM6 (SEQ ID NO: 41), SLC33A1 (SEQ ID NO: 11), TRIM32 (SEQ ID NO: 37), ALG10 (SEQ ID NO: 27), TFCP2 (SEQ ID NO: 49), SERPINH1 (SEQ ID NO: 51), SELL (SEQ ID NO: 55), ZNF510 (SEQ ID NO: 53), or antigenic fragments thereof, attached to the support.

Using a sequential screening strategy to select antigen-specific antibodies (AAb) from 4988 tumor antigens, 119 AAb potential novel biomarkers for the early detection of breast cancer were identified. A blinded validation study produced supporting evidence for 28 of these potential biomarkers, recited above. Thus, the polypeptide probe sets of the invention can be used, for example, to detect tumor antigen-specific autoantibodies in a bodily fluid sample from patients with breast cancer, such as early stage breast cancer. Descriptions of the polypeptides, their amino acid sequences and their nucleic acid sequences are provided in Table 1.

In various embodiments, the polypeptide probe sets comprise at least 2, 3, 4, 5, 6, 7, 8, 9, 10, 11, 12, 13, 14, 15, 16, 17, 18, 19, 20, 21, 22, 23, 24, 25, 26, 27, or all 28 of the recited polypeptides, or antigenic portions thereof.

In a preferred embodiment, the at least 2 different isolated polypeptides in the probe sets are selected from the group consisting of ATP6AP1 (SEQ ID NO: 13), PDCD6IP (SEQ ID NO: 21), DBT (SEQ ID NO: 25), CSNKIE (SEQ ID NO: 9), FRS3 (SEQ ID NO: 3), HOXD1 (SEQ ID NO: 7), SF3A1 (SEQ ID NO: 1), C15orf48 (SEQ ID NO: 35), MYOZ2 (SEQ ID NO: 33), BAT4 (SEQ ID NO: 5), BMX (SEQ ID NO: 45), RAB5A (SEQ ID NO: 23), UBAP1 (SEQ ID NO: 47), GPR157 (SEQ ID NO: 43), ZMYM6 (SEQ ID NO: 41), SLC33A1 (SEQ ID NO: 11), TRIM32 (SEQ ID NO: 37), ALG10 (SEQ ID NO: 27), TFCP2 (SEQ ID NO: 49), SERPINH1 (SEQ ID NO: 51), SELL (SEQ ID NO: 55), ZNF510 (SEQ ID NO: 53), or antigenic fragments thereof. Thus, in various embodiments, the polypeptide probe sets comprise at least 2, 3, 4, 5, 6, 7, 8, 9, 10, 11, 12, 13, 14, 15, 16, 17, 18, 19, 20, 21, or all 22 of the recited polypeptides, or antigenic portions thereof. In a further preferred embodiment the probe sets comprise ATP6AP1 (SEQ ID NO: 13) and at least one other of the recited polypeptides, or antigenic portions thereof. Thus, in various embodiments, the polypeptide arrays comprise at least ATP6AP1 and 1, 2, 3, 4, 5, 6, 7, 8, 9, 10, 11, 12, 13, 14, 15, 16, 17, 18, 19, 20, 21, 22, 23, 24, 25, 26, or all 27 of the other recited polypeptides, or antigenic portions thereof.

In a preferred embodiment, the probe sets comprise at least 2, 3, 4, 5, or all 6 of ATP6AP1 (SEQ ID NO: 13), CTBP1 (SEQ ID NO: 29), EIF3E (SEQ ID NO: 39), ATF3 (SEQ ID NO: 19), SOX2 (SEQ ID NO: 31), and BDNF (SEQ ID NO: 17), or antigenic portions thereof. The term “polypeptide” is used in its broadest sense to refer to a polymer of subunit amino acids, amino acid analogs, or peptidomimetics, including proteins and peptoids. The polypeptides may be naturally occurring full length proteins or fragments thereof, processed forms of naturally occurring polypeptides (such as by enzymatic digestion), chemically synthesized polypeptides, or recombinantly expressed polypeptides. The polypeptides may comprise D- and/or L-amino acids, as well as any other synthetic amino acid subunit, and may contain any other type of suitable modification, including but not limited to peptidomimetic bonds and reduced peptide bonds.

As used herein, an “antigenic fragment” is any portion of at least 4 amino acids of the recited polypeptide that can give rise to an immune response. In various preferred embodiments, the antigenic fragments are at least 5, 6, 7, 8, 9, 10, 11, 12, 13, 14, 151, 16, 17, 18, 19, 20, 21, 22, 23, 24, 25, 26, 27, 28, 29, 30, 35, 40, 45, 50, 75, 100, 150, 200, 250, 300, or the full amino acid sequence of the recited polypeptide.

In various further preferred embodiments, that can be combined with any other embodiments, the polypeptide probe sets comprise no more than 20,000 different polypeptides, or antigenic portions thereof and preferably comprise no more than 10,000; 5,000; 1,000; 500; 250; 100; 75; 50; 45; 40; 35; 30; 28, 27, 26, 25, 24, 23, 22, 21, 20, 19, 18, 17, 16, 15, 14, 13, 12, 11, 10, 9, 8, 7, 6, 5, 4, 3, or 2 different polypeptides. In this and other embodiments, two or more antigenic portions of the same polypeptide in the probe set count only as 1 polypeptide or antigenic portion thereof.

As will be appreciated by those of skill in the art, it may be desirable to include further polypeptides or other molecules in the probe sets as references, controls, positional markers, or as additional markers. Any suitable such further polypeptide or other molecule can be used. Exemplary additional polypeptide markers include but are not limited to p53, CTBP1, RAC3, and activating transcription factor-3 (ATF3). Exemplary analytical controls include human IgG and empty spots (when the probe set is present on a support). Any portion or the entirety of the recited polypeptides may be used in the probe set, so long as it is capable of binding to autoantibodies against the polypeptide.

The polypeptide probe sets can be present in any form useful for a given purpose. In various preferred embodiments, they can be present in solution, lyophilized, frozen, or immobilized on a substrate.

In one preferred embodiment, the polypeptides are immobilized on a substrate. Any suitable technique for immobilizing the polypeptides on the support can be used. In one embodiment, Nucleic Acid Protein Programmable Array (NAPPA technology can be used. NAPPA arrays are generated by printing full-length cDNAs encoding the target proteins at each feature of the array. The proteins are then transcribed and translated by a cell-free system and immobilized in situ using epitope tags fused to the proteins. Other suitable immobilization methods include, but are not limited to luciferase immunoprecipitation systems (LIPS), Luminex™ beads, wells of a 96 well dish, standard immune dipstick assays, standard ELISA assays,

As used herein, an array may be any arrangement or disposition of the polypeptides. In one embodiment, the polypeptides are at specific and identifiable locations on the array. Those of skill in the art will recognize that many such permutations of the polypeptides on the array are possible. In another non-limiting embodiment, each distinct location on the array comprises a distinct polypeptide.

Any suitable support may be used. Examples of such supports include, but are not limited to, microarrays, beads, columns, optical fibers, wipes, nitrocellulose, nylon, glass, quartz, diazotized membranes (paper or nylon), silicones, polyformaldehyde, cellulose, cellulose acetate, paper, ceramics, metals, metalloids, semiconductive materials, coated beads, magnetic particles; plastics such as polyethylene, polypropylene, and polystyrene; and gel-forming materials, such as proteins (e.g., gelatins), lipopolysaccharides, silicates, agarose, polyacrylamides, methylmethacrylate polymers; sol gels; porous polymer hydrogels; nanostructured surfaces; nanotubes (such as carbon nanotubes), and nanoparticles (such as gold nanoparticles or quantum dots). In one embodiment, the support is a solid support. Any suitable “solid support” may be used to which the polypeptides can be attached including but not limited to dextrans, hydrogels, silicon, quartz, other piezoelectric materials such as langasite (La₃Ga₅SiO₁₄), nitrocellulose, nylon, glass, diazotized membranes (paper or nylon), polyformaldehyde, cellulose, cellulose acetate, paper, ceramics, metals, metalloids, semiconductive materials, coated beads, magnetic particles; plastics such as polyethylene, polypropylene, and polystyrene; and gel-forming materials, such as proteins (e.g., gelatins), lipopolysaccharides, silicates, agarose and polyacrylamides.

Any suitably sized solid support can be used. In one non-limiting example, the solid support comprises slides with dimensions of approximately 3 inches by 1 inch.

In all embodiments of the invention, the polypeptides of the probe set may further comprise a tag, such as a detectable moiety. This is particularly preferred when the polypeptide probe sets or in solution, or in any other format where different polypeptides in the probe set cannot be distinguished by differential positions on a support. In such embodiments, it is particularly preferred that the different polypeptides, or antigenic fragments thereof, that are present in the probe set are distinguishable, through the use of differentially detectable tags, using techniques known to those of skill in the art. The tag(s) can be linked to the polypeptide through covalent bonding, including, but not limited to, disulfide bonding, hydrogen bonding, electrostatic bonding, recombinant fusion and conformational bonding. Alternatively, the tag(s) can be linked to the polypeptide by means of one or more linking compounds. Techniques for conjugating tags to polypeptides are well known to the skilled artisan. The polypeptides of the probe set, comprising a detectable tag can be used diagnostically to, for example, assess the presence of antibodies to the polypeptides in a sample; and thereby detect the presence of breast cancer, or monitor the development or progression of breast cancer as part of a clinical testing procedure. Any suitable detection tag can be used, including but not limited to enzymes, prosthetic groups, fluorescent materials, luminescent materials, bioluminescent materials, radioactive materials, positron emitting metals, and nonradioactive paramagnetic metal ions. The tag used will depend on the specific detection/analysis/diagnosis techniques and/or methods used such as immunohistochemical staining of (tissue) samples, flow cytometric detection, scanning laser cytometric detection, fluorescent immunoassays, enzyme-linked immunosorbent assays (ELISAs), radioimmunoassays (RIAs), bioassays (e.g., neutralization assays), Western blotting applications, etc. For immunohistochemical staining of tissue samples preferred tags are enzymes that catalyze production and local deposition of a detectable product. Enzymes typically conjugated to polypeptides to permit their immunohistochemical visualization are well known and include, but are not limited to, acetylcholinesterase, alkaline phosphatase, beta-galactosidase, glucose oxidase, horseradish peroxidase, and urease. Typical substrates for production and deposition of visually detectable products are also well known to the skilled person in the art. The polypeptides can be labeled using colloidal gold or they can be labeled with radioisotopes, such as 33p, 32p, 35S, 3H, and 1251. Polypeptides of the probe set can be attached to radionuclides directly or indirectly via a chelating agent by methods well known in the art.

In a second aspect, the present invention provides polynucleotide arrays comprising:
(a) a support; and
(b) at least 2 different isolated nucleic acids encoding polypeptides selected from the group consisting of ATP6AP1 (SEQ ID NO: 14), PDCD6IP (SEQ ID NO: 22), DBT (SEQ ID NO: 26), CSNKIE (SEQ ID NO: 10), FRS3 (SEQ ID NO: 4), RAC3 (SEQ ID NO: 16), HOXD1 (SEQ ID NO: 8), SF3A1 (SEQ ID NO: 2), CTBP1 (SEQ ID NO: 30), C15orf48 (SEQ ID NO: 36), MYOZ2 (SEQ ID NO: 34), EIF3E (SEQ ID NO: 40), BAT4 (SEQ ID NO: 6), ATF3 (SEQ ID NO: 20), BMX (SEQ ID NO: 46), RAB5A (SEQ ID NO: 24), UBAP1 (SEQ ID NO: 48), SOX2 (SEQ ID NO: 32), GPR157 (SEQ ID NO: 44), BDNF (SEQ ID NO: 18), ZMYM6 (SEQ ID NO: 42), SLC33A1 (SEQ ID NO: 12), TRIM32 (SEQ ID NO: 38), ALG10 (SEQ ID NO: 28), TFCP2 (SEQ ID NO: 50), SERPINH1 (SEQ ID NO: 52), SELL (SEQ ID NO: 56), ZNF510 (SEQ ID NO: 54), or antigenic fragments thereof, attached to the support. In this aspect, the arrays can also be used for example, to detect tumor antigen-specific autoantibodies in patients with breast cancer, such as early stage breast cancer. Any suitable technique can be used for attaching the nucleic acids to the support. In one embodiment, NAPPA arrays are generated by printing fcDNAs encoding the target proteins, or antigenic fragments thereof, at features of the support. Other techniques for printing nucleic acids on a support can be used and are well known in the art.

In various embodiments, the arrays comprise at least 2, 3, 4, 5, 6, 7, 8, 9, 10, 11, 12, 13, 14, 15, 16, 17, 18, 19, 20, 21, 22, 23, 24, 25, 26, 27, or all 28 of the recited nucleic acids, attached to the support.

In a preferred embodiment the at least 2 different isolated nucleic acids encode polypeptides selected from the group consisting of ATP6AP1 (SEQ ID NO: 14), PDCD6IP (SEQ ID NO: 22), DBT (SEQ ID NO: 26), CSNKIE (SEQ ID NO: 10), FRS3 (SEQ ID NO: 4), HOXD1 (SEQ ID NO: 8), SF3A1 (SEQ ID NO: 2), C15orf48 (SEQ ID NO: 36), MYOZ2 (SEQ ID NO: 34), BAT4 (SEQ ID NO: 6), BMX (SEQ ID NO: 46), RAB5A (SEQ ID NO: 24), UBAP1 (SEQ ID NO: 48), GPR157 (SEQ ID NO: 44), ZMYM6 (SEQ ID NO: 42), SLC33A1 (SEQ ID NO: 12), TRIM32 (SEQ ID NO: 38), ALG10 (SEQ ID NO: 28), TFCP2 (SEQ ID NO: 50), SERPINH1 (SEQ ID NO: 52), SELL (SEQ ID NO: 56), ZNF510 (SEQ ID NO: 54), or antigenic fragments thereof. Thus, in various embodiments, the polynucleotide arrays comprise at least 2, 3, 4, 5, 6, 7, 8, 9, 10, 11, 12, 13, 14, 15, 16, 17, 18, 19, 20, 21, or all 22 of the recited nucleic acids, or antigenic portions thereof, attached to the support.

In a further preferred embodiment the at least 2 different isolated nucleic acids encode ATP6AP1 (SEQ ID NO: 14) and at least one other of the recited nucleic acids, or antigenic portions thereof. Thus, in various embodiments, the polynucleotide arrays comprise at least ATP6AP1 and 1, 2, 3, 4, 5, 6, 7, 8, 9, 10, 11, 12, 13, 14, 15, 16, 17, 18, 19, 20, 21, 22, 23, 24, 25, 26, or all 27 of the other recited nucleic acids, or antigenic portions thereof, attached to the support.

In another preferred embodiment, the at least isolated nucleic acids encode 2, 3, 4, 5, or all 6 of ATP6AP1 (SEQ ID NO: 13), CTBP1 (SEQ ID NO: 29), EIF3E (SEQ ID NO: 39), ATF3 (SEQ ID NO: 19), SOX2 (SEQ ID NO: 31), and BDNF (SEQ ID NO: 17), or antigenic portions thereof.

In various further preferred embodiments, that can be combined with any other embodiments, the arrays comprise no more than 20,000 different nucleic acids, and preferably comprise no more than 10,000; 5,000; 1,000; 500; 250; 100; 75; 50; 45; 40; 35; 30; 28, 27, 26, 25, 24, 23, 22, 21, 20, 19, 18, 17, 16, 15, 14, 13, 12, 11, 10, 9, 8, 7, 6, 5, 4, 3, or 2 different nucleic acids.

As will be appreciated by those of skill in the art, it may be desirable to place nucleic acids encoding other polypeptides on the support as controls, positional markers, or as additional markers, including but not limited to p53, CTBP1, RAC3, and activating transcription factor-3 (ATF3).

Any portion or the entirety of the recited nucleic acid may be attached to the support, so long as it is encodes a polypeptide, or antigenic fragment thereof, capable of binding to autoantibodies against the polypeptide.

The definitions and all embodiments disclosed in the first aspect apply to this second aspect.

In a third aspect, the present invention provides methods for detecting breast cancer, comprising;
(a) contacting a suitable bodily fluid sample obtained from a subject at risk of breast cancer with one or more isolated polypeptides selected from the group consisting of ATP6AP1 (SEQ ID NO: 13), PDCD6IP (SEQ ID NO: 21), DBT (SEQ ID NO: 25), CSNKIE (SEQ ID NO: 9), FRS3 (SEQ ID NO: 3), RAC3 (SEQ ID NO: 15), HOXD1 (SEQ ID NO: 7), SF3A1 (SEQ ID NO: 1), CTBP1 (SEQ ID NO: 29), C15orf48 (SEQ ID NO: 35), MYOZ2 (SEQ ID NO: 33), EIF3E (SEQ ID NO: 39), BAT4 (SEQ ID NO: 5), ATF3 (SEQ ID NO: 19), BMX (SEQ ID NO: 45), RAB5A (SEQ ID NO: 23), UBAP1 (SEQ ID NO: 47), SOX2 (SEQ ID NO: 31), GPR157 (SEQ ID NO: 43), BDNF (SEQ ID NO: 17), ZMYM6 (SEQ ID NO: 41), SLC33A1 (SEQ ID NO: 11), TRIM32 (SEQ ID NO: 37), ALG10 (SEQ ID NO: 27), TFCP2 (SEQ ID NO: 49), SERPINH1 (SEQ ID NO: 51), SELL (SEQ ID NO: 55), ZNF510 (SEQ ID NO: 53), or antigenic fragments thereof; wherein the contacting occurs under conditions suitable for selective binding of antibodies in the bodily fluid sample to the one or more polypeptides; and
(b) detecting presence of antibodies to the polypeptides in the bodily fluid sample;
wherein the presence of antibodies in the bodily fluid sample to the one or more polypeptides indicates a likelihood of breast cancer in the subject.

The inventors have discovered that the presence of autoantibodies to one or more of the recited polypeptides is a positive predictor of breast cancer, and thus the methods of the invention provide valuable diagnostic and prognostic information to an attending physician.

As used herein a subject “at risk of breast cancer” is any human considered to be in a risk group for breast cancer. In one embodiment, the subject is a woman. In other embodiments, the subject has one or more of a lump in their breast tissue, lymph nodes, or armpit; changes in breast size or shape; skin dimpling; nipple inversion; spontaneous single-nipple discharge; a family/personal history of breast cancer; or is a carrier of a mutation in the BRCA or other gene that predisposes one to breast cancer.

Suitable bodily fluid samples include serum, plasma, CSF, pleural fluid, joint fluid, nipple discharge, saliva. In a preferred embodiment, the bodily fluid sample is serum or plasma.

In one embodiment, the presence of any amount of antibodies to the polypeptides in a sample from a subject at risk of breast cancer can indicate a likelihood of breast cancer in the subject. In another embodiment, if antibodies to the polypeptides are present in a sample from a subject at risk of breast cancer, at levels which are higher than that of a control sample (i.e. a sample from a subject who does not have breast cancer) than the subject at risk of breast cancer has a likelihood of breast cancer. Subjects with a likelihood of breast cancer can then be tested for the actual presence of breast cancer using standard diagnostic techniques known to the skilled artisan, including mammography, biopsy, or breast MRI. In various embodiments, the method results in an accurate diagnosis in at least 70% of cases; more preferably of at least 75%, 80%, 85%, 90%, or more of the cases. In a preferred embodiment, the likelihood of breast cancer is a likelihood of Stage I or Stage II breast cancer.

In various embodiments, the methods comprise contacting a bodily fluid sample, such as serum, obtained from a subject at risk of breast cancer with 2, 3, 4, 5, 6, 7, 8, 9, 10, 11, 12, 13, 14, 15, 16, 17, 18, 19, 20, 21, 22, 23, 24, 25, 26, 27, or all 28 of the recited polypeptides, or antigenic fragments thereof. In various embodiments, the presence of antibodies in the bodily fluid sample to 1, 2, 3, 4, 5, 6, 7, 8, 9, 10, 11, 12, 13, 14, 15, 16, 17, 18, 19, 20, 21, 22, 23, 24, 25, 26, 27, or all 28 of the recited polypeptides, or antigenic fragments thereof, indicates a likelihood of breast cancer in the subject.

In a preferred embodiment, the one or more isolated polypeptides are selected from the group consisting of ATP6AP1 (SEQ ID NO: 13), PDCD6IP (SEQ ID NO: 21), DBT (SEQ ID NO: 25), CSNKIE (SEQ ID NO: 9), FRS3 (SEQ ID NO: 3), HOXD1 (SEQ ID NO: 7), SF3A1 (SEQ ID NO: 1), C15orf48 (SEQ ID NO: 35), MYOZ2 (SEQ ID NO: 33), BAT4 (SEQ ID NO: 5), BMX (SEQ ID NO: 45), RAB5A (SEQ ID NO: 23), UBAP1 (SEQ ID NO: 47), GPR157 (SEQ ID NO: 43), ZMYM6 (SEQ ID NO: 41), SLC33A1 (SEQ ID NO: 11), TRIM32 (SEQ ID NO: 37), ALG10 (SEQ ID NO: 27), TFCP2 (SEQ ID NO: 49), SERPINH1 (SEQ ID NO: 51), SELL (SEQ ID NO: 55), ZNF510 (SEQ ID NO: 53), or antigenic fragments thereof. Thus, in various embodiments, the methods comprise contacting a serum sample obtained from a subject at risk of breast cancer with 2, 3, 4, 5, 6, 7, 8, 9, 10, 11, 12, 13, 14, 15, 16, 17, 18, 19, 20, 21, or all 22 of the recited polypeptides, or antigenic fragments thereof.

In a further preferred embodiment, the methods comprise contacting a bodily fluid sample, such as a serum sample, obtained from a subject at risk of breast cancer with ATP6AP1 (SEQ ID NO: 13), or an antigenic fragment thereof. In this embodiment, the method may further comprise contacting the serum sample with 1, 2, 3, 4, 5, 6, 7, 8, 9, 10, 11, 12, 13, 14, 15, 16, 17, 18, 19, 20, 21, 22, 23, 24, 25, 26, or all 27 of the other recited polypeptides, or antigenic portions thereof.

In a preferred embodiment, the methods comprise contacting a bodily fluid sample, such as a serum sample, obtained from a subject at risk of breast cancer with 2, 3, 4, 5, or all 6 of ATP6AP1 (SEQ ID NO: 13), CTBP1 (SEQ ID NO: 29), EIF3E (SEQ ID NO: 39), ATF3 (SEQ ID NO: 19), SOX2 (SEQ ID NO: 31), and BDNF (SEQ ID NO: 17), or antigenic portions thereof.

In one preferred embodiment, the method comprises contacting the bodily fluid sample, such as a serum sample, to a polypeptide array of any embodiment of the first aspect of the invention, or to an array according to any embodiment of the second aspect of the invention after the encoded proteins are then transcribed and translated by a cell-free system and immobilized in situ using epitope tags fused to the proteins.

As will be appreciated by those of skill in the art, it may be desirable to test for autoantibodies to other polypeptides, and so the method may comprise testing for such further autoantibodies, such as antibodies to p53, CTBP1, RAC3, and activating transcription factor-3 (ATF3).

The “binding” may comprise any detectable interaction of an antibody with an antigen (polypeptide or polynucleotide molecule), including without limitation a covalent bond, ionic bond, salt bridge, hydrogen bond, van der Waals interaction, hydrophobic/hydrophilic interaction, electrostatic interaction, steric interaction, other associations, or any combination of any of the foregoing. As will be understood by those of skill in the art, array interactions do not require chemical binding.

In one embodiment, a probe set, such as an array according to any embodiment of the invention are contacted with the bodily fluid, such as a serum sample, under conditions suitable for binding of antibodies in the fluid to antigens in the probe set; unbound antibodies are washed and bound antibodies are detected by labeled secondary reagents, such as labeled secondary antibodies. Suitable conditions and reagents to promote binding of specific antibody types to antigens (polypeptides or polynucleotides molecules) is well within the level of those of skill in the art. Thus, the methods of the invention are not limited by any specific type of binding conditions employed. Such conditions will vary depending on the type of sample, desired stringency of the binding interaction, and nature of the competing materials in the binding solution, the type of molecules (polypeptide or polynucleotide) in the probe set, the type of probe set, and, for embodiments in which the probe set is present on a support, the type of support, and the density of the molecules arrayed on the support. In a preferred embodiment, the conditions comprise a step to remove unbound antibodies. Determining the need for such a step, and appropriate conditions for such a step, are well within the level of skill in the art.

Any type of labeled secondary reagents label can be used in the methods of the present invention, including but not limited to radioisotope labels, fluorescent labels, luminescent labels, and electrochemical labels (i.e.: antibody labels with different electrode mid-point potential, where detection comprises detecting electric potential of the label). In a preferred embodiment, fluorescent or electrochemical labels are used. Detection of signal from detectable labels is well within the level of skill in the art. For example, fluorescent array readers are well known in the art, as are instruments to record electric potentials on a substrate (For electrochemical detection see, for example, J. Wang (2000) Analytical Electrochemistry, Vol., 2nd ed., Wiley-VCH, New York). In a further embodiment, the detectable labels comprise quantum dots. In one embodiment, secondary labels can be used, including but not limited to secondary antibodies or ligands that bind to the antibodies. In embodiments where multiple polypeptides are used as probes, it is preferable that they are differentially distinguishable, as discussed above. In a further embodiment, antibodies bound to each polypeptide are quantified by staining with anti-fusion tag antibodies and measurement of the fluorescence intensity signal generated from secondary antibodies. Detecting presence of antibodies to the polypeptides in the bodily fluid sample can be accomplished by standard methods in the art. Suitable conditions and reagents will be understood by those of skill in the art based on the teachings herein. The presence of antibodies to the polypeptides may be determined by immunoassay methods utilizing the antibodies described above. Such immunoassay methods include, but are not limited to, direct or indirect immunoassay such as for example a competitive binding assay, a non-competitive binding assay, a radioimmunoassay, immunohistochemistry, an enzyme-linked immunosorbent assay (ELISA), a sandwich assay, a gel diffusion immunodiffusion assay, an agglutination assay, dot blotting, a fluorescent immunoassay such as fluorescence-activated cell sorting (FACS), chemiluminescence immunoassay, immunoPCR immunoassay, a protein A or protein G immunoassay, and an immunoelectrophoresis assay such as western blotting and others commonly used and widely described in scientific and patent literature, and many employed commercially.

In the case of an enzyme immunoassay, an enzyme is conjugated to the second antibody, usually by means of glutaraldehyde or periodate. As will be readily recognized, however, a wide variety of different ligation techniques exist which are well-known to the skilled artisan. Commonly used enzymes include horseradish peroxidase, glucose oxidase, beta-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates, which yield a fluorescent product, rather than the chromogenic substrates noted above. A solution containing the appropriate substrate is then added to the tertiary complex. The substrate reacts with the enzyme linked to the second antibody, giving a qualitative visual signal, which may be further quantitated, usually spectrophotometrically, to give an evaluation of the amount of secreted protein or fragment thereof. Alternately, fluorescent compounds, such as fluorescein and rhodamine, may be chemically coupled to antibodies without altering their binding capacity. When activated by illumination with light of a particular wavelength, the fluorochrome-labeled antibody absorbs the light energy, inducing a state of excitability in the molecule, followed by emission of the light at a characteristic longer wavelength. The emission appears as a characteristic color visually detectable with a light microscope. Immunofluorescence and EIA techniques are both very well established in the art and are particularly preferred for the present method. However, other reporter molecules, such as radioisotopes, chemiluminescent or bioluminescent molecules may also be employed.

In a further embodiment, the presence of antibodies to the polypeptides may be determined by using Western blot analysis. The technique generally comprises separating sample antibody proteins by gel electrophoresis on the basis of molecular weight and transferring the antibody proteins to a suitable solid support, such as nitrocellulose filter, a nylon filter, or derivatized nylon filter. The sample is incubated with the polypeptides or antigenic fragments thereof that specifically bind the sample antibodies and the resulting complex is detected. The polypeptides may be directly labeled or alternatively may be subsequently detected using labeled secondary antibodies that specifically bind to the polypeptide-antibody complex. Antibody binding reagents may be, for example, protein A, or other antibodies. Antibody binding reagents may be radiolabeled or enzyme linked. Detection may be by autoradiography, calorimetric reaction or chemiluminescence. This method allows both quantitation of an amount of sample antibody and determination of its identity by a relative position on the membrane which is indicative of a migration distance in the acrylamide gel during electrophoresis. The definitions and all embodiments disclosed in the first and second aspects apply to this third aspect.

### EXAMPLES

Custom NAPPA protein microarrays were used to detect tumor antigen-specific AAb in the sera of patients with early-stage breast cancer. Using a sequential screening strategy to select AAb from 4988 tumor antigens, we identified 119 AAb potential biomarkers for the early detection of breast cancer. A blinded validation study produced supporting evidence for 28 of these potential biomarkers.

Sera used in these analyses were obtained from Fox Chase Cancer Center (FCCC) and the Duke University Medical Center (DUMC) with support from the NCI Early Detection Research Network and the NCI Breast SPORE program. Sera were derived from early-stage breast cancer patients from FCCC (53 cases/53 controls); control sera were sex- and age-matched (+/−2 yrs). All samples were obtained at the time of routine mammography, prior to the diagnosis of cancer, and were selected retrospectively. To control for benign breast disease, we obtained an independent set of sera of early-stage invasive breast cancer patients and age-matched (+/−3 yrs) benign breast disease controls from DUMC (102 cases/102 controls). These samples were collected using a standardized sample collection protocol and stored at −80° C. until use. Cases and matched controls were processed simultaneously. Written consent was obtained from all subjects under institutional review board approval.

Sequence-verified, full-length cDNA expression plasmids in flexible donor vector systems were obtained from the Harvard Institute of Proteomics and are publicly available (see web site dnasu.asu.edu/DNASU/). These were converted to the T7-based mammalian expression vector pANT7_GST using LR recombinase (Invitrogen, Carlsbad, CA). Expression plasmids were transformed into E. coli DH5α and grown in 1.5 mL terrific broth and ampicillin (100 μg/mL). DNA was purified with the NucleoPrepII anion exchange resin (Macherey-Nagel Inc., Bethlehem, PA) using a Biomek FX (Beckman Coulter, Inc., Fullerton, CA) automated laboratory workstation. Automated addition of all solutions was accomplished using a Matrix WellMate™ (Thermo Scientific, Hudson, NH) rapid bulk liquid-dispensing instrument. Purified DNA was precipitated by addition of 0.6 volumes isopropanol, followed by centrifugation at 5000 rcf for 30 minutes. The DNA pellet was washed with 200 μL of 80% ethanol, centrifuged at 5000 rcf for 15 minutes, dried, and resuspended in dH₂O. For bead array ELISAs, larger quantities of DNA were prepared using standard Nucleobond™ preparation methods (Macherey-Nagel Inc., Bethlehem, PA).

Plasmid DNA (1.5 μg/□μL) was supplemented with capture antibody (50 μg/mL, anti-GST antibody, GE Healthcare Biosciences, Piscataway, NJ) or anti-FLAG antibody (Sigma-Aldrich, St. Louis, MO), protein crosslinker (2 mM, BS3, Pierce, Rockford, IL) and BSA (3 mg/mL, Sigma-Aldrich) to the DNA prior to printing onto the array surface. All samples were printed using a Genetix QArray2™ with 300 μm solid tungsten pins on amine-treated glass slides. Arrays were stored in an air-tight container at room temperature, protected from light. The printed DNA was transcribed and translated in situ using previously published protocols. Protein expression was detected using anti-GST MAb (Cell Signaling, Danvers, MA) diluted at 1:200. For detecting serum antibodies, the arrays were incubated with serum diluted 1:300-1:600 in 5% PBS milk with 0.2% Tween 20. All incubations were carried out at 4° C. overnight with mixing (Corning hybridization chambers) unless indicated otherwise. Detection on the array was carried out using an anti-human IgG (Jackson ImmunoResearch Labs, West Grove, PA) conjugated with HRP. The slides were developed for fluorescent detection using the Tyramide Signal Amplification reagent (PerkinElmer, Waltham, MA) per manufacturer's instructions. Slides were scanned with a Perkin Elmer ProScanArray HT and the images were quantitated using Micro Vigene software (Vigene Tech version 2.9.9.2). The highly immunogenic EBV-derived antigen, EBNA-1, was included as N- and C-terminal fragments for positive control antigens. Negative controls included empty vectors and no DNA controls. Registration spots for array alignment were printed purified human IgG proteins.

For the first screening stage, 53 cases and 53 control sera from FCCC were screened on 4,988 antigens displayed in NAPPA protein array format. Each array was normalized by first removing the background signal estimated by the first quartile of the non-spots and then log-transforming the median-scaled raw intensities to bring the data to the same scale and stabilize the variance across the range of signals. Candidate antigens from the initial 4,988 antigens were selected if they met two different criteria: 1) comparison of the 95^{th} percentiles of the cases and controls using quantile regression and 2) comparison of the proportion of cases with intensities above the 95^{th} percentile of controls to the expected number seen by chance, with a p-value≤0.05 (n=217). Additional antigens (n=544) were ranked based on intensity and decreasing specificity (cases/controls). Independent arrays of these 761 candidate antigens were screened with a fully independent set of age-matched sera consisting of 76 controls with benign breast disease and 102 patient sera from DUMC, randomly divided into training and validation sets. We normalized these arrays as follows. First, we removed differences in intensity associated with plates and pins by consecutively multiplying the raw intensities by three factors: the median intensity of all antigens divided by the median intensity of antigens from the same plate, the median intensity of all antigens divided by the median intensity of antigens printed at the same within-pin position, and the median intensity of all antigens divided by the median intensity of antigens printed with the same pin. These scalings yielded a median reduction in variance of 9%. We removed any duplicate antigen pairs that differed by more than 3 times the median absolute deviation, resulting in removal of 0.5% of spots. Third, we rescaled the raw intensities as above and averaged duplicate antigen pairs. Finally, we removed background signal by subtracting the first quartile of control spot (no DNA) intensity and divided the excess intensity by the median excess intensity.

We used the partial area under the receiver operating characteristic curve (pAUC) as the basis for comparing the normalized intensities of cases and controls for each antigen. Specifically, we used the pAUC where the false positive rate is at most 5%. For each antigen we tested the hypothesis that the pAUC was greater than 0.00125, which is the same partial area under the 45 degree line receiver operating characteristic curve that represents no difference between cases and controls. We used the training set to identify 119 potential antigen biomarkers with p-values less than 0.05 and confirmed 28 of these using the validation set (p<0.05). Training and validation statistics for the 28 breast cancer biomarkers is provided in Table 1.

TABLE 1

| Gene Name | Accession number | Amino acid | Nucleotide |
|---|---|---|---|
| SF3A1 | NP_005868 | SEQ ID NO: 1 | SEQ ID NO: 2 |
| splicing factor 3A | | | |
| subunit 1 isoform 1- | | | |
| full length (1-793) | | | |
| FRS3 | NP_006644 | SEQ ID NO: 3 | SEQ ID NO: 4 |
| fibroblast growth | | | |
| factor receptor | | | |
| substrate 3-full | | | |
| length (1-492) | | | |
| BAT4 | NP_149417 | SEQ ID NO: 5 | SEQ ID NO: 6 |
| HLA-B associated | | | |
| transcript-4-full | | | |
| length (1-356) | | | |
| HOXD1 | AAH14477 | SEQ ID NO: 7 | SEQ ID NO: 8 |
| homeobox protein | | | |
| Hox-D1 full length | | | |
| (1-328) | | | |
| CSNK1E | NP_001885 | SEQ ID NO: 9 | SEQ ID NO: 10 |
| casein kinase I | | | |
| isoform epsilon full | | | |
| length (1-416) | | | |
| SLC33A1 | NP_004724 | SEQ ID NO: 11 | SEQ ID NO: 12 |
| acetyl-coenzyme A | | | |
| transporter 1-full | | | |
| length (1-549) | | | |
| ATP6AP1 | NP_001174 | SEQ ID NO: 13 | SEQ ID NO: 14 |
| V-type proton | | | |
| ATPase subunit S1 | | | |
| precursor-full length | | | |
| (1-470) | | | |
| RAC3 | NP_005043 | SEQ ID NO: 15 | SEQ ID NO: 16 |
| ras-related C3 | | | |
| botulinum toxin | | | |
| substrate 3 | | | |
| precursor-full length | | | |
| (1-192) | | | |
| BDNF | AAA96140 | SEQ ID NO: 17 | SEQ ID NO: 18 |
| brain-derived | | | |
| neurotrophic factor | | | |
| transcript variant 5- | | | |
| full length (1-247) | | | |
| ATF3 | NP_001665 | SEQ ID NO: 19 | SEQ ID NO: 20 |
| cyclic AMP- | | | |
| dependent | | | |
| transcription factor | | | |
| ATF-3 isoform 1-full | | | |
| length (1-181) | | | |
| PDCD6IP | NP_037506 | SEQ ID NO: 21 | SEQ ID NO: 22 |
| programmed cell | | | |
| death 6-interacting | | | |
| protein isoform 1- | | | |
| full length (1-868) | | | |
| RAB5A | NP_004153 | SEQ ID NO: 23 | SEQ ID NO: 24 |
| ras-related protein | | | |
| Rab-5A-full length | | | |
| (1-215) | | | |
| DBT | AAH16675 | SEQ ID NO: 25 | SEQ ID NO: 26 |
| Dihydrolipoamide | | | |
| branched chain | | | |
| transacylase E2-full | | | |
| length (1-482) | | | |
| ALG10 | NP_116223 | SEQ ID NO: 27 | SEQ ID NO: 28 |
| alpha-1,2- | | | |
| glucosyltransferase | | | |
| ALG10-A-full length | | | |
| (1-473) | | | |
| CTBP1 | NP_001319 | SEQ ID NO: 29 | SEQ ID NO: 30 |
| C-terminal-binding | | | |
| protein 1 isoform 1- | | | |
| full length (1-440) | | | |
| SOX2 | NP_003097 | SEQ ID NO: 31 | SEQ ID NO: 32 |
| transcription factor | | | |
| SOX-2-full length | | | |
| (1-317) | | | |
| MYOZ2 | NP_057683 | SEQ ID NO: 33 | SEQ ID NO: 34 |
| myozenin-2-full | | | |
| length (1-264) | | | |
| C15orf48 | NP_115789 | SEQ ID NO: 35 | SEQ ID NO: 36 |
| normal mucosa of | | | |
| esophagus-specific | | | |
| gene 1 protein-full | | | |
| length (1-83) | | | |
| TRIM32 | NP_001093149 | SEQ ID NO: 37 | SEQ ID NO: 38 |
| E3 ubiquitin-protein | | | |
| ligase TRIM32-full | | | |
| length (1-653) | | | |
| EIF3E | NP_001559 | SEQ ID NO: 39 | SEQ ID NO: 40 |
| eukaryotic | | | |
| translation initiation | | | |
| factor 3 subunit E- | | | |
| full length (1-445) | | | |
| ZMYM6 | AAP35781 | SEQ ID NO: 41 | SEQ ID NO: 42 |
| zinc finger, MYM- | | | |
| type 6, isoform | | | |
| CRA_b-partial (1- | | | |
| 156/163) | | | |
| GPR157 | EAW71612 | SEQ ID NO: 43 | SEQ ID NO: 44 |
| probable G-protein | | | |
| coupled receptor | | | |
| 157-partial (1- | | | |
| 155/335) | | | |
| BMX | NP_001712 | SEQ ID NO: 45 | SEQ ID NO: 46 |
| cytoplasmic | | | |
| tyrosine-protein | | | |
| kinase BMX-full | | | |
| length (1-675) | | | |
| UBAP1 | NP_057609 | SEQ ID NO: 47 | SEQ ID NO: 48 |
| ubiquitin-associated | | | |
| protein 1 isoform 1- | | | |
| full length (1-502) | | | |
| TFCP2 | AAH36890 | SEQ ID NO: 49 | SEQ ID NO: 50 |
| grainyhead-like 3 | | | |
| (Drosophila), | | | |
| isoform CRA_d-full | | | |
| length (1-555) | | | |
| SERPINH1 | NP_001226 | SEQ ID NO: 51 | SEQ ID NO: 52 |
| serpin H1 precursor- | | | |
| full length (1-418) | | | |
| ZNF510 | AAH68587 | SEQ ID NO: 53 | SEQ ID NO: 54 |
| zinc finger protein | | | |
| 510-partial (1- | | | |
| 636/683) | | | |
| SELL | AAH20758 | SEQ ID NO: 55 | SEQ ID NO: 56 |
| L-selectin | | | |
| precursor-full length | | | |
| (1-375) | | | |

1-18. (canceled)

19. A method comprising:
(a) contacting a serum or plasma sample obtained from a subject having, or at risk of having, breast cancer with a polynucleotide probe set bound to a surface of a solid support, wherein the polynucleotide probe set comprises two or more isolated polynucleotides selected from the group consisting of ATP6AP1 (SEQ ID NO: 14), PDCD6IP (SEQ ID NO: 22), DBT (SEQ ID NO: 26), CSNK1E (SEQ ID NO: 10), FRS3 (SEQ ID NO: 4), HOXD1 (SEQ ID NO: 8), SF3A1 (SEQ ID NO: 2), C15orf48 (SEQ ID NO: 36), MYOZ2 (SEQ ID NO: 34), BAT4 (SEQ ID NO: 6), BMX (SEQ ID NO: 46), RAB5A (SEQ ID NO: 24), UBAP1 (SEQ ID NO: 48), GPR157 (SEQ ID NO: 44), ZMYM6 (SEQ ID NO: 42), SLC33A1 (SEQ ID NO: 12), TRIM32 (SEQ ID NO: 38), ALG10 (SEQ ID NO: 28), TFCP2 (SEQ ID NO: 50), SERPINH1 (SEQ ID NO: 52), SELL (SEQ ID NO: 56), ZNF510 (SEQ ID NO: 54) or antigenic fragments thereof; wherein the contacting occurs under conditions whereby antibodies, if present in the bodily fluid, selectively bind to the polynucleotides bound to the surface;
(b) detecting presence of antibodies to the polynucleotides in the sample; wherein the presence of antibodies in the sample to the two or more polynucleotides is used to diagnose breast cancer in the subject, and wherein detecting comprises normalizing intensities for each polynucleotide of the probe set and comparing normalized intensities with a control using partial area under a receiver operating characteristic curve (pAUC) with a false positive rate no greater than 5 percent; and
(c) administering a therapeutic agent to the subject to treat the breast cancer.

20. The method of claim 1, wherein the one or more isolated polynucleotide comprises ATP6AP1 (SEQ ID NO: 13), or an antigenic fragment thereof.

21. The method of claim 1 wherein the subject has one or more of a lump in the breast tissue, lymph nodes, or armpit; changes in breast size or shape, skin dimpling, nipple inversion, spontaneous single-nipple discharge, a family/personal history of breast cancer, or the subject is a carrier of a mutation in the BRCA or other gene that predisposes the subject to breast cancer.

22. The method of claim 1, wherein the breast cancer is Stage I or Stage II breast cancer.

23. The method of claim 1, wherein the detecting comprises use of a labeled antibody.

24. The method of claim 1, wherein the subject is further tested for the presence of breast cancer using a technique selected from the group consisting of mammography, biopsy, or magnetic resonance imaging.

25. The method of claim 1, wherein the polypeptide probe set further comprises two or more additional polypeptides selected from the group recited in claim 1.

26. The method of claim 1, wherein each polynucleotide of the polynucleotide probe set is bound to the surface using Nucleic Acid Programmable Protein Array (NAPPA) technology.


SEQUENCE LISTING (56 sequences)

SEQ ID NO: 1 - protein, 793 aa, Homo sapiens
MPAGPVQAVPPPPPVPTEPKQPTEEEASSKEDSAPSKPVVGIIYPPPEVRNIVDKTASFVARNGPEFEARIRQNEINNPKFNFLNPNDPYHAYYRHKVSEFKEGKAQEPSAAIPKVMQQQQQTTQQQLPQKVQAQVIQETIVPKEPPPEFEFIADPPSISAFDLDVVKLTAQFVARNGRQFLTQLMQKEQRNYQFDFLRPQHSLFNYFTKLVEQYTKILIPPKGLFSKLKKEAENPREVLDQVCYRVEWAKFQERERKKEEEEKEKERVAYAQIDWHDFVVVETVDFQPNEQGNFPPPTTPEELGARILIQERYEKFGESEEVEMEVESDEEDDKQEKAEEPPSQLDQDTQVQDMDEGSDDEEEGQKVPPPPETPMPPPLPPTPDQVIVRKDYDPKASKPLPPAPAPDEYLVSPITGEKIPASKMQEHMRIGLLDPRWLEQRDRSIREKQSDDEVYAPGLDIESSLKQLAERRTDIFGVEETAIGKKIGEEEIQKPEEKVTWDGHSGSMARTQQAAQANITLQEQIEAIHKAKGLVPEDDTKEKIGPSKPNEIPQQPPPPSSATNIPSSAPPITSVPRPPTMPPPVRTTVVSAVPVMPRPPMASVVRLPPGSVIAPMPPIIHAPRINVVPMPPSAPPIMAPRPPPMIVPTAFVPAPPVAPVPAPAPMPPVHPPPPMEDEPTSKKLKTEDSLMPEEEFLRRNKGPVSIKVQVPNMQDKTEWKLNGQVLVFTLPLTDQVSVIKVKIHEATGMPAGKQKLQYEGIFIKDSNSLAYYNMANGAVIHLALKERGGRKK

SEQ ID NO: 2 - DNA, 2379 bp, Homo sapiens
  source 1..2379: mol_type = unassigned DNA
atgccggccggacccgtgcaggcggtgcccccgccgccgcccgtgcccacggagcccaaacagcccacagaagaagaagcatcttcaaaggaggattctgcaccttctaagccagttgtggggattatttaccctcctccagaggtcagaaatattgttgacaagactgccagctttgtggccagaaacgggcctgaatttgaagctaggatccgacagaacgagatcaacaaccccaagttcaactttctgaaccccaatgacccttaccatgcctactaccgccacaaggtcagcgagttcaaggaagggaaggctcaggagccgtccgccgccatccccaaggtcatgcagcagcagcagcagaccacccagcagcagctgccccagaaggtccaagcccaagtaatccaagagaccatcgtgcccaaagagcctcctcctgagtttgagttcattgctgatcctccctctatctcagccttcgacttggatgtggtgaagctgacggctcagtttgtggccaggaatgggcgccagtttctgacccagctgatgcagaaagagcagcgcaactaccagtttgactttctccgcccacagcacagcctcttcaactacttcacgaagctagtggaacagtacaccaagatcttgattccacccaaaggtttattttcaaagctcaagaaagaggctgaaaacccccgagaagttttggatcaggtgtgttaccgagtggaatgggccaaattccaggaacgtgagaggaagaaggaagaagaggagaaggagaaggagcgggtggcctatgctcagatcgactggcatgattttgtggtggtggaaacagtggacttccaacccaatgagcaagggaacttccctccccccaccacgccagaggagctgggggcccgaatcctcattcaggagcgctatgaaaagtttggggagagtgaggaagttgagatggaggtcgagtctgatgaggaggatgacaaacaggagaaggcggaggagcctccttcccagctggaccaggacacccaagtacaagatatggatgagggttcagatgatgaagaagaagggcagaaagtgcccccacccccagagacacccatgcctccacctctgcccccaactccagaccaagtcattgtccgcaaggattatgatcccaaagcctccaagcccttgcctccagcccctgctccagatgagtatcttgtgtcccccattactggggagaagatccccgccagcaaaatgcaggaacacatgcgcattggacttcttgaccctcgctggctggagcagcgggatcgctccatccgtgagaagcagagcgatgatgaggtgtacgcaccaggtctggatattgagagcagcttgaagcagttggctgagcggcgtactgacatcttcggtgtagaggaaacagccattggtaagaagatcggtgaggaggagatccagaagccagaggaaaaggtgacctgggatggccactcaggcagcatggcccggacccagcaggctgcccaggccaacatcaccctccaggagcagattgaggccattcacaaggccaaaggcctggtgccagaggatgacactaaagagaagattggccccagcaagcccaatgaaatccctcaacagccaccgccaccatcttcagccaccaacatccccagctcggctccacccatcacttcagtgccccgaccacccacaatgccacctccagttcgtactacagttgtctccgcagtacccgtcatgccccggcccccaatggcatctgtggtccggctgcccccaggctcagtgatcgcccccatgccgcccatcatccacgcgcccagaatcaacgtggtgcccatgcctccctcggcccctcctattatggccccccgcccaccccccatgattgtgccaacagcctttgtgcctgctccacctgtggcacctgtcccagctccagccccaatgccccctgtgcatcccccacctcccatggaagatgagcccacctccaaaaaactgaagacagaggacagcctcatgccagaggaggagttcctgcgcagaaacaagggtccagtgtccatcaaagtccaggtgcccaacatgcaggataagacggaatggaaactgaatgggcaggtgctggtcttcaccctcccactcacggaccaggtctctgtcattaaggtgaagattcatgaagccacaggcatgcctgcagggaaacagaagctacagtatgagggtatcttcatcaaagattccaactcactggcttactacaacatggccaatggcgcagtcatccacctggccctcaaggagagaggcgggaggaagaag

SEQ ID NO: 3 - protein, 492 aa, Homo sapiens
MGSCCSCLNRDSVPDNHPTKFKVTNVDDEGVELGSGVMELTQSELVLHLHRREAVRWPYLCLRRYGYDSNLFSFESGRRCQTGQGIFAFKCSRAEEIFNLLQDLMQCNSINVMEEPVIITRNSHPAELDLPRAPQPPNALGYTVSSFSNGCPGEGPRFSAPRRLSTSSLRHPSLGEESTHALIAPDEQSHTYVNTPASEDDHRRGRHCLQPLPEGQAPFLPQARGPDQRDPQVFLQPGQVKFVLGPTPARRHMVKCQGLCPSLHDPPHHNNNNEAPSECPAQPKCTYENVTGGLWRGAGWRLSPEEPGWNGLAHRRAALLHYENLPPLPPVWESQAQQLGGEAGDDGDSRDGLTPSSNGFPDGEEDETPLQKPTSTRAAIRSHGSFPVPLTRRRGSPRVFNFDFRRPGPEPPRQLNYIQVELKGWGGDRPKGPQNPSSPQAPMPTTHPARSSDSYAVIDLKKTVAMSNLQRALPRDDGTARKTRHNSTDLPL

SEQ ID NO: 4 - DNA, 1476 bp, Homo sapiens
  source 1..1476: mol_type = unassigned DNA
atggggagctgctgcagctgcctgaacagagacagcgttccagacaaccaccccaccaagttcaaggtgacaaatgtggatgatgagggggtggagctgggctctggggtgatggagctgacgcagagtgagctggtgctgcacctgcatcggcgtgaggccgtccgctggccttatctctgcttgcggcgctatggctacgactccaacctcttctcctttgagagtggccgccgatgtcagacaggccagggaatatttgcatttaagtgttcccgggctgaggaaatcttcaacctccttcaggatctgatgcagtgcaacagcatcaatgtgatggaagagcctgtcatcatcacccgcaatagccaccccgctgagcttgacctccctcgagccccccagccacccaatgctctaggctacactgtctccagcttttccaatggctgccctggagagggcccacgattctcagctccccggcggctctcgacaagcagcctgcggcacccctcgcttggggaagagtccacccatgccctcattgctcctgatgagcagtcccacacctatgtcaacacaccggccagtgaagatgaccaccgcaggggccgccactgcctgcagcccctgcctgagggtcaggcacccttcctcccgcaggcccggggacctgaccaacgggacccacaggtgttcttgcagccaggccaggtgaagtttgtgttgggcccgacccctgctcggcggcacatggtgaagtgccagggcctctgtcccagcctgcatgaccccccacaccacaataataacaatgaggccccttctgagtgtccagcccagcccaagtgcacctacgagaacgtcaccggggggctgtggcgaggggctggctggagactgagcccagaggagccgggctggaatggccttgcccaccgccgggccgccctgctgcactatgagaacctgcccccactgccccctgtgtgggaaagccaagcccagcagctgggaggggaggctggggatgatggggactcgagggatgggctcacaccctcttccaatggcttccctgatggtgaggaggacgagaccccactgcagaagcccaccagcacccgggccgccatccgcagccacggcagctttcctgtgccactgacccgccgccgcggctccccaagggtcttcaactttgatttccgccggccggggcccgagcccccaaggcagcttaactacatccaggtggagctaaagggctggggtggagaccgccctaaggggccccagaacccctcgagcccccaagcccccatgcccaccacccaccctgcccgaagctcagactcctacgccgtgattgacctcaaaaagaccgtggccatgtccaacctgcagagagctctgccccgagacgatggcaccgccaggaaaacccggcacaacagcaccgacctgcctctg

SEQ ID NO: 5 - protein, 356 aa, Homo sapiens
MSRPLLITFTPATDPSDLWKDGQQQPQPEKPESTLDGAAARAFYEALIGDESSAPDSQRSQTEPARERKRKKRRIMKAPAAEAVAEGASGRHGQGRSLEAEDKMTHRILRAAQEGDLPELRRLLEPHEAGGAGGNINARDAFWWTPLMCAARAGQGAAVSYLLGRGAAWVGVCELSGRDAAQLAEEAGFPEVARMVRESHGETRSPENRSPTPSLQYCENCDTHFQDSNHRTSTAHLLSLSQGPQPPNLPLGVPISSPGFKLLLRGGWEPGMGLGPRGEGRANPIPTVLKRDQEGLGYRSAPQPRVTHFPAWDTRAVAGRERPPRVATLSWREERRREEKDRAWERDLRTYMNLEF

SEQ ID NO: 6 - DNA, 1068 bp, Homo sapiens
  source 1..1068: mol_type = unassigned DNA
atgtcccggcccttgctcatcaccttcaccccagccactgaccccagcgacctctggaaggatgggcagcagcagccacagcccgagaagccagagtccaccctggatggggctgcagcccgagctttctatgaggccctgattggggatgagagcagcgctcctgactcccagagatctcagactgaacctgccagagaaagaaagagaaagaaaagaagaataatgaaggcaccagcagcagaagcagtggcagaaggagcatcaggaagacatggacaagggagatcccttgaggctgaggataagatgactcaccggatactgagggcagcccaggagggggacctgccagaacttaggagactgctggaaccgcatgaggcaggaggagctggggggaatatcaacgcccgggatgccttctggtggaccccactgatgtgtgctgctcgagcgggccagggggcagctgtgagctatctcctgggccgtggggctgcctgggtgggggtctgtgagctgagtggcagggatgcggctcagctcgctgaagaagctggcttccctgaggtagcccgcatggtcagggagagccatggagagacaaggagcccggaaaaccggtctcctactccctccctccagtactgcgagaactgtgacacccacttccaagattccaaccaccgcacatccactgctcacctgctgtcactgtcgcagggtcctcagcctcccaaccttccacttggggtgcccatctccagcccgggcttcaaactgctgctgagggggggctgggagccaggaatggggctgggaccccggggtgagggccgtgccaatcccatccccactgtcctcaagagggaccaggaaggactaggctacagatcagcaccccagccccgagtgacacatttcccagcttgggatacccgagctgtggctgggagggagagaccccctcgggtggccacactgagctggagggaggagagaaggagggaggagaaagacagggcttgggagcgggatctaaggacttacatgaacctcgagttc

SEQ ID NO: 7 - protein, 328 aa, Homo sapiens [sequence omitted]

SEQ ID NO: 8 - DNA, 984 bp, Homo sapiens
  source 1..984: mol_type = unassigned DNA
atgagctcctacctggagtacgtgtcatgcagcagcagcggcggggtcggcggcgacgtgctcagcttggcacccaagttctgccgctccgacgcccggcccgtggctctgcagcccgccttccctctgggcaacggcgacggcgccttcgtcagctgtctgcccctggccgccgcccgaccctcgccttcgcccccggccgcccccgcgcggccgtccgtaccgcctccggccgcgccccagtacgcgcagtgcaccctggagggggcctacgaacctggtgccgcacctgccgcggcagctgggggcgcggactacggcttcctggggtccgggccggcgtacgacttcccgggcgtgctggggcgggcggccgacgacggcgggtctcacgtccactacgccacctcggccgtcttctcgggcggcggctctttcctcctcagcggccaggtggattacgcggccttcggcgaacccggccctttttcggcttgtctcaaagcgtcagccgacggccaccctggtgctttccagaccgcatccccggccccaggcacctaccccaagtccgtctctcccgcctccggcctccctgccgccttcagcacgttcgagtggatgaaagtgaagaggaatgcctctaagaaaggcaaactcgccgagtatggggccgctagcccctccagcgcgatccgcacgaatttcagcaccaagcaactgacagaactggaaaaagagtttcatttcaataagtacttaactcgagcccggcgcatcgagatagccaactgcttgcacctgaatgacacgcaagtcaaaatctggttccagaaccgcaggatgaaacagaagaaaagggaacgagaagggcttctggccacggccattcctgtggctcccctccaacttcccctctctggaacaacccccactaagtttatcaagaaccccggcagcccttctcagtcccaagagccttcg

SEQ ID NO: 9 - protein, 416 aa, Homo sapiens
MELRVGNKYRLGRKIGSGSFGDIYLGANIASGEEVAIKLECVKTKHPQLHIESKFYKMMQGGVGIPSIKWCGAEGDYNVMVMELLGPSLEDLFNFCSRKFSLKTVLLLADQMISRIEYIHSKNFIHRDVKPDNFLMGLGKKGNLVYIIDFGLAKKYRDARTHQHIPYRENKNLTGTARYASINTHLGIEQSRRDDLESLGYVLMYFNLGSLPWQGLKAATKRQKYERISEKKMSTPIEVLCKGYPSEFSTYLNFCRSLRFDDKPDYSYLRQLFRNLFHRQGFSYDYVFDWNMLKFGAARNPEDVDRERREHEREERMGQLRGSATRALPPGPPTGATANRLRSAAEPVASTPASRIQPAGNTSPRAISRVDRERKVSMRLHRGAPANVSSSDLTGRQEVSRIPASQTSVPFDHLGK

SEQ ID NO: 10 - DNA, 1248 bp, Homo sapiens
  source 1..1248: mol_type = unassigned DNA
atggagctacgtgtggggaacaagtaccgcctgggacggaagatcgggagcgggtccttcggagatatctacctgggtgccaacatcgcctctggtgaggaagtcgccatcaagctggagtgtgtgaagacaaagcacccccagctgcacatcgagagcaagttctacaagatgatgcagggtggcgtggggatcccgtccatcaagtggtgcggagctgagggcgactacaacgtgatggtcatggagctgctggggcctagcctcgaggacctgttcaacttctgttcccgcaaattcagcctcaagacggtgctgctcttggccgaccagatgatcagccgcatcgagtatatccactccaagaacttcatccaccgggacgtcaagcccgacaacttcctcatggggctggggaagaagggcaacctggtctacatcatcgacttcggcctggccaagaagtaccgggacgcccgcacccaccagcacattccctaccgggaaaacaagaacctgaccggcacggcccgctacgcttccatcaacacgcacctgggcattgagcaaagccgtcgagatgacctggagagcctgggctacgtgctcatgtacttcaacctgggctccctgccctggcaggggctcaaagcagccaccaagcgccagaagtatgaacggatcagcgagaagaagatgtcaacgcccatcgaggtcctctgcaaaggctatccctccgaattctcaacatacctcaacttctgccgctccctgcggtttgacgacaagcccgactactcttacctacgtcagctcttccgcaacctcttccaccggcagggcttctcctatgactacgtctttgactggaacatgctgaaattcggtgcagcccggaatcccgaggatgtggaccgggagcggcgagaacacgaacgcgaggagaggatggggcagctacgggggtccgcgacccgagccctgccccctggcccacccacgggggccactgccaaccggctccgcagtgccgccgagcccgtggcttccacgccagcctcccgcatccagccggctggcaatacttctcccagagcgatctcgcgggtcgaccgggagaggaaggtgagtatgaggctgcacaggggtgcgcccgccaacgtctcctcctcagacctcactgggcggcaagaggtctcccggatcccagcctcacagacaagtgtgccatttgaccatctcgggaag

SEQ ID NO: 11 - protein, 549 aa, Homo sapiens
MSPTISHKDSSRQRRPGNFSHSLDMKSGPLPPGGWDDSHLDSAGREGDREALLGDTGTGDFLKAPQSFRAELSSILLLLFLYVLQGIPLGLAGSIPLILQSKNVSYTDQAFFSFVFWPFSLKLLWAPLVDAVYVKNFGRRKSWLVPTQYILGLFMIYLSTQVDRLLGNTDDRTPDVIALTVAFFLFEFLAATQDIAVDGWALTMLSRENVGYASTCNSVGQTAGYFLGNVLFLALESADFCNKYLRFQPQPRGIVTLSDFLFFWGTVFLITTTLVALLKKENEVSVVKEETQGITDTYKLLFAIIKMPAVLTFCLLILTAKIGFSAADAVTGLKLVEEGVPKEHLALLAVPMVPLQIILPLIISKYTAGPQPLNTFYKAMPYRLLLGLEYALLVWWTPKVEHQGGFPIYYYIVVLLSYALHQVTVYSMYVSIMAFNAKVSDPLIGGTYMTLLNTVSNLGGNWPSTVALWLVDPLTVKECVGASNQNCRTPDAVELCKKLGGSCVTALDGYYVESIICVFIGFGWWFFLGPKFKKLQDEGSSSWKCKRNN

SEQ ID NO: 12 - DNA, 1647 bp, Homo sapiens
  source 1..1647: mol_type = unassigned DNA
atgtcacccaccatctcccacaaggacagcagccggcaacggcggccagggaatttcagtcactctctggatatgaagagcggtcccctgccgccaggcggttgggatgacagtcatttggactcagcgggccgggaaggggacagagaagctcttctgggggataccggcactggcgacttcttaaaagccccacagagcttccgggccgaactaagcagcattttgctactactctttctttacgtgcttcagggtattcccctgggcttggcgggaagcatcccactcattttgcaaagcaaaaatgttagctatacagaccaagctttcttcagttttgtcttttggcccttcagtctcaaattactctgggccccgttggttgatgcggtctacgttaagaacttcggtcgtcgcaaatcttggcttgtcccgacacagtatatactaggactcttcatgatctatttatccactcaggtggaccgtttgcttgggaataccgatgacagaacacccgacgtgattgctctcactgtggcgttctttttgtttgaattcttggccgccactcaggacattgccgtcgatggttgggcgttaactatgttatccagggaaaatgtgggttatgcttctacttgcaattcggtgggccaaacagcgggttactttttgggcaatgttttgtttttggcccttgaatctgccgacttttgtaacaaatatttgcggtttcagcctcaacccagaggaatcgttactctttcagatttcctttttttctggggaactgtatttttaataacaacaacattggttgcccttctgaaaaaagaaaacgaagtatcagtagtaaaagaagaaacacaagggatcacagatacttacaagctgctttttgcaattataaaaatgccagcagttctgacattttgccttctgattctaactgcaaagattggtttttcagcagcagatgctgtaacaggactgaaattggtagaagagggagtacccaaagaacatttagccttattggcagttccaatggttcctttgcagataatactgcctctgattatcagcaaatacactgcaggtccccagccattaaacacattttacaaagccatgccctacagattattgcttgggttagaatatgccctactggtttggtggactcctaaagtagaacatcaagggggattccctatatattactatatcgtagtcctgctgagttatgctttacatcaggttacagtgtacagcatgtatgtttctataatggctttcaatgcaaaggttagtgatccacttattggaggaacatacatgacccttttaaataccgtgtccaatctgggaggaaactggccttctacagtagctctttggcttgtagatcccctcacagtaaaagagtgtgtaggagcatcaaaccagaattgtcgaacacctgatgctgttgagctttgcaaaaaactgggtggctcatgtgttacagccctggatggttattatgtggagtccattatttgtgttttcattggatttggttggtggttctttcttggtccaaaatttaaaaagttacaggatgaaggatcatcttcgtggaaatgcaaaaggaacaat

SEQ ID NO: 13 - protein, 470 aa, Homo sapiens
MMAAMATARVRMGPRCAQALWRMPWLPVFLSLAAAAAAAAAEQQVPLVLWSSDRDLWAPAADTHEGHITSDLQLSTYLDPALELGPRNVLLFLQDKLSIEDFTAYGGVFGNKQDSAFSNLENALDLAPSSLVLPAVDWYAVSTLTTYLQEKLGASPLHVDLATLRELKLNASLPALLLIRLPYTASSGLMAPREVLTGNDEVIGQVLSTLKSEDVPYTAALTAVRPSRVARDVAVVAGGLGRQLLQKQPVSPVIHPPVSYNDTAPRILFWAQNFSVAYKDQWEDLTPLTFGVQELNLTGSFWNDSFARLSLTYERLFGTTVTFKFILANRLYPVSARHWFTMERLEVHSNGSVAYFNASQVTGPSIYSFHCEYVSSLSKKGSLLVARTQPSPWQMMLQDFQIQAFNVMGEQFSYASDCASFFSPGIWMGLLTSLFMLFIFTYGLHMILSLKTMDRFDDHKGPTISLTQIV

SEQ ID NO: 14 - DNA, 1410 bp, Homo sapiens
  source 1..1410: mol_type = unassigned DNA
atgatggcggccatggcgacggctcgagtgcggatggggccgcggtgcgcccaggcgctctggcgcatgccgtggctgccggtgtttttgtcgttggcggcggcggcggcggcggcagcggcggagcagcaggtcccgctggtgctgtggtcgagtgaccgggacttgtgggctcctgcggccgacactcatgaaggccacatcaccagcgacttgcagctctctacctacttagatcccgccctggagctgggtcccaggaatgtgctgctgttcctgcaggacaagctgagcattgaggatttcacagcatatggcggtgtgtttggaaacaagcaggacagcgccttttctaacctagagaatgccctggacctggccccctcctcactggtgcttcctgccgtcgactggtatgcagtcagcactctgaccacttacctgcaggagaagctcggggccagccccttgcatgtggacctggccaccctgcgggagctgaagctcaatgccagcctccctgctctgctgctcattcgcctgccctacacagccagctctggtctgatggcacccagggaagtcctcacaggcaacgatgaggtcatcgggcaggtcctgagcacactcaagtccgaagatgtcccatacacagcggccctcacagcggtccgcccttccagggtggcccgtgatgtagccgtggtggccggagggctaggtcgccagctgctacaaaaacagccagtatcacctgtgatccatcctcctgtgagttacaatgacaccgctccccggatcctgttctgggcccaaaacttctctgtggcgtacaaggaccagtgggaggacctgactcccctcacctttggggtgcaggaactcaacctgactggctccttctggaatgactcctttgccaggctctcactgacctatgaacgactctttggtaccacagtgacattcaagttcattctggccaaccgcctctacccagtgtctgcccggcactggtttaccatggagcgcctcgaagtccacagcaatggctccgtcgcctacttcaatgcttcccaggtcacagggcccagcatctactccttccactgcgagtatgtcagcagcctgagcaagaagggtagtctcctcgtggcccgcacgcagccctctccctggcagatgatgcttcaggacttccagatccaggctttcaacgtaatgggggagcagttctcctacgccagcgactgtgccagcttcttctcccccggcatctggatggggctgctcacctccctgttcatgctcttcatcttcacctatggcctgcacatgatcctcagcctcaagaccatggatcgctttgatgaccacaagggccccactatttctttgacccagattgtg

SEQ ID NO: 15 - protein, 192 aa, Homo sapiens
MQAIKCVVVGDGAVGKTCLLISYTTNAFPGEYIPTVFDNYSANVMVDGKPVNLGLWDTAGQEDYDRLRPLSYPQTDVFLICFSLVSPASFENVRAKWYPEVRHHCPHTPILLVGTKLDLRDDKDTIERLRDKKLAPITYPQGLAMAREIGSVKYLECSALTQRGLKTVFDEAIRAVLCPPPVKKPGKKCTVF

SEQ ID NO: 16 - DNA, 576 bp, Homo sapiens
  source 1..576: mol_type = unassigned DNA
atgcaggccatcaagtgcgtggtggtcggcgacggcgccgtggggaagacatgcttgctgatcagctacacgaccaacgccttccccggagagtacatccccaccgtttttgacaactactctgccaacgtgatggtggacgggaaaccagtcaacttggggctgtgggacacagcgggtcaggaggactacgatcggctgcggccactctcctacccccaaactgacgtctttctgatctgcttctctctggtgagcccggcctccttcgagaatgttcgtgccaagtggtacccggaggtgcggcaccactgcccccacacgcccatcctcctggtgggcaccaagctggacctccgcgacgacaaggacaccattgagcggctgcgggacaagaagctggcacccatcacctacccacagggcctggccatggcccgggagattggctctgtgaaatacctggagtgctcagccctgacccagcggggcctgaagacagtgtttgacgaggcgatccgcgcggtgctctgcccgcccccagtgaagaagccggggaagaagtgcaccgtcttc

SEQ ID NO: 17 - protein, 247 aa, Homo sapiens [sequence omitted]

SEQ ID NO: 18 - DNA, 741 bp, Homo sapiens
  source 1..741: mol_type = unassigned DNA
atgaccatccttttccttactatggttatttcatactttggttgcatgaaggctgcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacatgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagacgctgcaaacatgtccatgagggtccggcgccactctgaccctgcccgccgaggggagctgagcgtgtgtgacagtattagtgagtgggtaacggcggcagacaaaaagactgcagtggacatgtcgggcgggacggtcacagtccttgaaaaggtccctgtatcaaaaggccaactgaagcaatacttctacgagaccaagtgcaatcccatgggttacacaaaagaaggctgcaggggcatagacaaaaggcattggaactcccagtgccgaactacccagtcgtacgtgcgggcccttaccatggatagcaaaaagagaattggctggcgattcataaggatagacacttcttgtgtatgtacattgaccattaaaaggggaaga

SEQ ID NO: 19 - protein, 181 aa, Homo sapiens
MMLQHPGQVSASEVSASAIVPCLSPPGSLVFEDFANLTPFVKEELRFAIQNKHLCHRMSSALESVTVSDRPLGVSITKAEVAPEEDERKKRRRERNKIAAAKCRNKKKEKTECLQKESEKLESVNAELKAQIEELKNEKQHLIYMLNLHRPTCIVRAQNGRTPEDERNLFIQQIKEGTLQS

SEQ ID NO: 20 - DNA, 543 bp, Homo sapiens
  source 1..543: mol_type = unassigned DNA
atgatgcttcaacacccaggccaggtctctgcctcggaagtgagtgcttctgccatcgtcccctgcctgtcccctcctgggtcactggtgtttgaggattttgctaacctgacgccctttgtcaaggaagagctgaggtttgccatccagaacaagcacctctgccaccggatgtcctctgcgctggaatcagtcactgtcagcgacagacccctcggggtgtccatcacaaaagccgaggtagcccctgaagaagatgaaaggaaaaagaggcgacgagaaagaaataagattgcagctgcaaagtgccgaaacaagaagaaggagaagacggagtgcctgcagaaagagtcggagaagctggaaagtgtgaatgctgaactgaaggctcagattgaggagctcaagaacgagaagcagcatttgatatacatgctcaaccttcatcggcccacgtgtattgtccgggctcagaatgggaggactccagaagatgagagaaacctctttatccaacagataaaagaaggaacattgcagagc

SEQ ID NO: 21 - protein, 868 aa, Homo sapiens
MATFISVQLKKTSEVDLAKPLVKFIQQTYPSGGEEQAQYCRAAEELSKLRRAAVGRPLDKHEGALETLLRYYDQICSIEPKFPFSENQICLTFTWKDAFDKGSLFGGSVKLALASLGYEKSCVLFNCAALASQIAAEQNLDNDEGLKIAAKHYQFASGAFLHIKETVLSALSREPTVDISPDTVGTLSLIMLAQAQEVFFLKATRDKMKDAIIAKLANQAADYFGDAFKQCQYKDTLPKEVFPVLAAKHCIMQANAEYHQSILAKQQKKFGEEIARLQHAAELIKTVASRYDEYVNVKDFSDKINRALAAAKKDNDFIYHDRVPDLKDLDPIGKATLVKSTPVNVPISQKFTDLFEKMVPVSVQQSLAAYNQRKADLVNRSIAQMREATTLANGVLASLNLPAAIEDVSGDTVPQSILTKSRSVIEQGGIQTVDQLIKELPELLQRNREILDESLRLLDEEEATDNDLRAKFKERWQRTPSNELYKPLRAEGTNFRTVLDKAVQADGQVKECYQSHRDTIVLLCKPEPELNAAIPSANPAKTMQGSEVVNVLKSLLSNLDEVKKEREGLENDLKSVNFDMTSKFLTALAQDGVINEEALSVTELDRVYGGLTTKVQESLKKQEGLLKNIQVSHQEFSKMKQSNNEANLREEVLKNLATAYDNFVELVANLKEGTKFYNELTEILVRFQNKCSDIVFARKTERDELLKDLQQSIAREPSAPSIPTPAYQSSPAGGHAPTPPTPAPRTMPPTKPQPPARPPPPVLPANRAPSATAPSPVGAGTAAPAPSQTPGSAPPPQAQGPPYPTYPGYPGYCQMPMPMGYNPYAYGQYNMPYPPVYHQSPGQAPYPGPQQPSYPFPQPPQQSYYPQQ

SEQ ID NO: 22 - DNA, 2604 bp, Homo sapiens
  source 1..2604: mol_type = unassigned DNA
atggcgacattcatctcggtgcagctgaaaaagacctcagaggtggacctggccaagccgctggtgaagttcatccagcagacttacccaagcggcggggaagagcaggcccagtactgccgcgcggcggaggagctcagcaagctgcgccgcgccgcagtcggtcgtccgctggacaagcacgagggcgcgctcgagacgctcctgagatattatgatcagatttgttctattgaacccaaattcccattttctgaaaatcagatctgcttgacatttacctggaaggatgctttcgataaaggttcactttttggaggctctgtaaaactggctcttgcaagcttaggatatgaaaagagctgtgtgttgttcaattgtgcagccttagctagccaaattgcagcagaacagaacctggataatgatgaaggattgaaaatcgctgctaaacattaccagtttgctagtggtgcctttttacatattaaagagacggttttatctgccttaagtcgagagccgaccgtggacatatctccagatactgttgggaccctcagtcttattatgctggcacaggctcaagaagtattttttttaaaagccacaagagataaaatgaaagatgccatcatagctaaattggctaatcaggctgcagattattttggtgatgctttcaaacagtgtcaatacaaagatactctccccaaggaggtgttccctgtcttggctgcaaagcactgtatcatgcaggccaatgctgagtaccatcagtctatcctggcaaaacagcagaagaaatttggagaagaaattgcaaggttacagcatgcagcagaactgattaaaacagtggcatctcgctatgatgaatatgttaatgtgaaggatttttctgacaaaatcaatcgtgcccttgctgcagcaaagaaggataatgacttcatttatcatgatcgagttccagaccttaaagatctagatcctattggcaaagccacacttgtgaaatctaccccggtcaatgtacccatcagtcagaaatttactgatctgtttgagaagatggttcccgtgtcagtacagcagtctttggctgcctataatcagaggaaagccgatttggttaacagatcaattgctcagatgagagaagccaccactttggcaaatggggtgctagcttcccttaatcttccagcagcaattgaagatgtgtctggagacactgtacctcagtctatattgactaaatccagatctgtgattgaacagggaggcatccagactgttgatcagttgattaaagaactgcctgaattactgcaacgaaatagagaaatcctagatgagtcattaaggttgttggatgaagaagaagcaaccgataatgatttaagagcaaaatttaaggaacgttggcaaaggacaccatccaatgaactgtataagcctttaagagcagagggaaccaacttcagaacagttttagataaagctgtgcaggcagatggacaagtgaaagaatgttaccagtctcatcgtgacaccatcgtgcttttgtgtaagccagagcctgagctgaatgctgccatcccttctgctaatccagcaaagaccatgcagggcagtgaggttgtaaatgtcttaaaatccttattgtcaaatcttgatgaagtaaagaaggaaagagagggtctggagaatgacttgaaatctgtgaattttgacatgacaagcaagtttttgacagccctggctcaagatggtgtgataaatgaagaagctctttctgttactgaactagatcgagtctatggaggtcttacaactaaagtccaagaatctctaaagaaacaggagggacttcttaaaaatattcaggtctcacatcaggaattttcaaaaatgaaacaatctaataatgaagctaacttaagagaagaagttttgaagaatttagctactgcatatgacaactttgttgaacttgtagctaatttgaaggaaggcacaaagttttacaatgagttgactgaaatcctggtcaggttccagaacaaatgcagtgatatagtttttgcacggaagacagaaagagatgaactcttaaaggacttgcaacaaagcattgccagagaacctagtgctccttcaattcctacacctgcgtatcagtcctcaccagcaggaggacatgcaccaactcctccaactccagcgccaagaaccatgccgcctactaagccccagcccccagccaggcctccaccacctgtgcttccagcaaatcgagctccttctgctactgctccatctccagtgggggctgggactgctgcgccagctccatcacaaacgcctggctcagctcctcctccacaggcgcagggaccaccctatcccacctatccaggatatcctgggtattgccaaatgcccatgcccatgggctataatccttatgcgtatggccagtataatatgccatatccaccagtgtatcaccagagtcctggacaggctccatacccgggaccccagcagccttcataccccttccctcagcccccacagcagtcttactatccacagcag

SEQ ID NO: 23 - protein, 215 aa, Homo sapiens
MASRGATRPNGPNTGNKICQFKLVLLGESAVGKSSLVLRFVKGQFHEFQESTIGAAFLTQTVCLDDTTVKFEIWDTAGQERYHSLAPMYYRGAQAAIVVYDITNEESFARAKNWVKELQRQASPNIVIALSGNKADLANKRAVDFQEAQSYADDNSLLFMETSAKTSMNVNEIFMAIAKKLPKNEPQNPGANSARGRGVDLTEPTQPTRNQCCSN

SEQ ID NO: 24 - DNA, 645 bp, Homo sapiens
  source 1..645: mol_type = unassigned DNA
atggctagtcgaggcgcaacaagacccaacgggccaaatactggaaataaaatatgccagttcaaactagtacttctgggagagtccgctgttggcaaatcaagcctagtgcttcgttttgtgaaaggccaatttcatgaatttcaagagagtaccattggggctgcttttctaacccaaactgtatgtcttgatgacactacagtaaagtttgaaatatgggatacagctggtcaagaacgataccatagcctagcaccaatgtactacagaggagcacaagcagccatagttgtatatgatatcacaaatgaggagtcctttgcaagagcaaaaaattgggttaaagaacttcagaggcaagcaagtcctaacattgtaatagctttatcgggaaacaaggccgacctagcaaataaaagagcagtagatttccaggaagcacagtcctatgcagatgacaatagtttattattcatggagacatccgctaaaacatcaatgaatgtaaatgaaatattcatggcaatagctaaaaaattgccaaagaatgaaccacaaaatccaggagcaaattctgccagaggaagaggagtagaccttaccgaacccacacaaccaaccaggaatcagtgttgtagtaac

SEQ ID NO: 25 - protein, 482 aa, Homo sapiens
MAAVRMLRTWSRNAGKLICVRYFQTCGNVHVLKPNYVCFFGYPSFKYSHPHHFLKTTAALRGQVVQFKLSDIGEGIREVTVKEWYVKEGDTVSQFDSICEVQSDKASVTITSRYDGVIKKLYYNLDDIAYVGKPLVDIETEALKDSEEDVVETPAVSHDEHTHQEIKGRKTLATPAVRRLAMENNIKLSEVVGSGKDGRILKEDILNYLEKQTGAILPPSPKVEIMPPPPKPKDMTVPILVSKPPVFTGKDKTEPIKGFQKAMVKTMSAALKIPHFGYCDEIDLTELVKLREELKPIAFARGIKLSFMPFFLKAASLGLLQFPILNASVDENCQNITYKASHNIGIAMDTEQGLIVPNVKNVQICSIFDIATELNRLQKLGSVGQLSTTDLTGGTFTLSNIGSIGGTFAKPVIMPPEVAIGALGSIKAIPRFNQKGEVYKAQIMNVSWSADHRVIDGATMSRFSNLWKSYLENPAFMLLDLK

SEQ ID NO: 26 - DNA, 1446 bp, Homo sapiens
  source 1..1446: mol_type = unassigned DNA
atggctgcagtccgtatgctgagaacctggagcaggaatgcggggaagctgatttgtgttcgctattttcaaacatgtggtaatgttcatgttttgaagccaaattatgtgtgtttctttggttatccttcattcaagtatagtcatccacatcacttcctgaaaacaactgctgctctccgtggacaggttgttcagttcaagctctcagacattggagaagggattagagaagtaactgttaaagaatggtatgtaaaagaaggagatacagtgtctcagtttgatagcatctgtgaagttcaaagtgataaagcttctgttaccatcactagtcgttatgatggagtcattaaaaaactctattataatctagacgatattgcctatgtggggaagccattagtagacatagaaacggaagctttaaaagattcagaagaagatgttgttgaaactcctgcagtgtctcatgatgaacatacacaccaagagataaagggccgaaaaacactggcaactcctgcagttcgccgtctggcaatggaaaacaatattaagctgagtgaagttgttggctcaggaaaagatggcagaatacttaaagaagatatcctcaactatttggaaaagcagacaggagctatattgcctccttcacccaaagttgaaattatgccacctccaccaaagccaaaagacatgactgttcctatactagtatcaaaacctccggtattcacaggcaaagacaaaacagaacccataaaaggctttcaaaaagcaatggtcaagactatgtctgcagccctgaagatacctcattttggttattgtgatgagattgaccttactgaactggttaagctccgagaagaattaaaacccattgcatttgctcgtggaattaaactctcctttatgcctttcttcttaaaggctgcttccttgggattactacagtttcctatccttaacgcttctgtggatgaaaactgccagaatataacatataaggcttctcataacattgggatagcaatggatactgagcagggtttgattgtccctaatgtgaaaaatgttcagatctgctctatatttgacatcgccactgaactgaaccgcctccagaaattgggctctgtgggtcagctcagcaccactgatcttacaggaggaacatttactctttccaacattggatcaattggtggtacctttgccaaaccagtgataatgccacctgaagtagccattggggcccttggatcaattaaggccattccccgatttaaccagaaaggagaagtatataaggcacagataatgaatgtgagctggtcagctgatcacagagttattgatggtgctacaatgtcacgcttctccaatttgtggaaatcctatttagaaaacccagcttttatgctactagatctgaaa

SEQ ID NO: 27 - protein, 473 aa, Homo sapiens
MAQLEGYYFSAALSCTFLVSCLLFSAFSRALREPYMDEIFHLPQAQRYCEGHFSLSQWDPMITTLPGLYLVSIGVIKPAIWIFGWSEHVVCSIGMLRFVNLLFSVGNFYLLYLLFCKVQPRNKAASSIQRVLSTLTLAVFPTLYFFNFLYYTEAGSMFFTLFAYLMCLYGNHKTSAFLGFCGFMFRQTNIIWAVFCAGNVIAQKLTEAWKTELQKKEDRLPPIKGPFAEFRKILQFLLAYSMSFKNLSMLLLLTWPYILLGFLFCAFVVVNGGIVIGDRSSHEACLHFPQLFYFFSFTLFFSFPHLLSPSKIKTFLSLVWKRRILFFVVTLVSVFLVWKFTYAHKYLLADNRHYTFYVWKRVFQRYETVKYLLVPAYIFAGWSIADSLKSKSIFWNLMFFICLFTVIVPQKLLEFRYFILPYVIYRLNIPLPPTSRLICELSCYAVVNFITFFIFLNKTFQWPNSQDIQRFMW

SEQ ID NO: 28 - DNA, 1419 bp, Homo sapiens
  source 1..1419: mol_type = unassigned DNA
atggcgcagctggaaggttactatttctcggccgccttgagctgtacctttttagtatcctgcctcctcttctccgccttcagccgggcgttgcgagagccctacatggacgagatcttccacctgcctcaggcgcagcgctactgtgagggccatttctccctttcccagtgggatcccatgattactacattacctggcttgtacctggtgtcaattggagtgatcaaacctgccatttggatctttggatggtctgaacatgttgtctgctccattgggatgctcagatttgttaatcttctcttcagtgttggcaacttctatttactatatttgcttttctgcaaggtacaacccagaaacaaggctgcctcaagtatccagagagtcttgtcaacattaacactagcagtatttccaacactttatttttttaacttcctttattatacagaagcaggatctatgttttttactctttttgcgtatttgatgtgtctttatggaaatcataaaacttcagccttccttggattttgtggcttcatgtttcggcaaacaaatatcatctgggctgtcttctgtgcaggaaatgtcattgcacaaaagttaacggaggcttggaaaactgagctacaaaagaaggaagacagacttccacctattaaaggaccatttgcagaattcagaaaaattcttcagtttcttttggcttattccatgtcctttaaaaacttgagtatgcttttgcttctgacttggccctacatccttctgggatttctgttttgtgcttttgtagtagttaatggtggaattgttattggcgatcggagtagtcatgaagcctgtcttcattttcctcaactattctactttttttcatttactctctttttttcctttcctcatctcctgtctcctagcaaaattaagacttttctttccttagtttggaaacgtagaattctgttttttgtggttaccttagtctctgtgtttttagtttggaaattcacttatgctcataaatacttgctagcagacaatagacattatactttctatgtgtggaaaagagtttttcaaagatatgaaactgtaaaatatttgttagttccagcctatatatttgctggttggagtatagctgactcattgaaatcaaagtcaattttttggaatttaatgtttttcatatgcttgttcactgttatagttcctcagaaactgctggaatttcgttacttcattttaccttatgtcatttataggcttaacatacctctgcctcccacatccagactcatttgtgaactgagctgctatgcagttgttaatttcataacttttttcatctttctgaacaagacttttcagtggccaaatagtcaggacattcaaaggtttatgtgg

SEQ ID NO: 29 - protein, 440 aa, Homo sapiens
MGSSHLLNKGLPLGVRPPIMNGPLHPRPLVALLDGRDCTVEMPILKDVATVAFCDAQSTQEIHEKVLNEAVGALMYHTITLTREDLEKFKALRIIVRIGSGFDNIDIKSAGDLGIAVCNVPAASVEETADSTLCHILNLYRRATWLHQALREGTRVQSVEQIREVASGAARIRGETLGIIGLGRVGQAVALRAKAFGFNVLFYDPYLSDGVERALGLQRVSTLQDLLFHSDCVTLHCGLNEHNHHLINDFTVKQMRQGAFLVNTARGGLVDEKALAQALKEGRIRGAALDVHESEPFSFSQGPLKDAPNLICTPHAAWYSEQASIEMREEAAREIRRAITGRIPDSLKNCVNKDHLTAATHWASMDPAVVHPELNGAAYRYPPGVVGVAPTGIPAAVEGIVPSAMSLSHGLPPVAHPPHAPSPGQTVKPEADRDHASDQL

SEQ ID NO: 30 - DNA, 1320 bp, Homo sapiens
  source 1..1320: mol_type = unassigned DNA
atgggcagctcgcacttgctcaacaagggcctgccgcttggcgtccgacctccgatcatgaacgggcccctgcacccgcggcccctggtggcattgctggatggccgggactgcacagtggagatgcccatcctgaaggacgtggccactgtggccttctgcgacgcgcagtccacgcaggagatccatgagaaggtcctgaacgaggctgtgggggccctgatgtaccacaccatcactctcaccagggaggacctggagaagttcaaagccctccgcatcatcgtccggattggcagtggttttgacaacatcgacatcaagtcggccggggatttaggcattgccgtctgcaacgtgcccgcggcgtctgtggaggagacggccgactcgacgctgtgccacatcctgaacctgtaccggcgggccacctggctgcaccaggcgctgcgggagggcacacgagtccagagcgtcgagcagatccgcgaggtggcgtccggcgctgccaggatccgcggggagaccttgggcatcatcggacttggtcgcgtggggcaggcagtggcgctgcgggccaaggccttcggcttcaacgtgctcttctacgacccttacttgtcggatggcgtggagcgggcgctggggctgcagcgtgtcagcaccctgcaggacctgctcttccacagcgactgcgtgaccctgcactgcggcctcaacgagcacaaccaccacctcatcaacgacttcaccgtcaagcagatgagacaaggggccttcctggtgaacacagcccggggtggcctggtggatgagaaggcgctggcccaggccctgaaggagggccggatccgcggcgcggccctggatgtgcacgagtcggaacccttcagctttagccagggccctctgaaggatgcacccaacctcatctgcaccccccatgctgcatggtacagcgagcaggcatccatcgagatgcgagaggaggcggcacgggagatccgcagagccatcacaggccggatcccagacagcctgaagaactgtgtcaacaaggaccatctgacagccgccacccactgggccagcatggaccccgccgtcgtgcaccctgagctcaatggggctgcctataggtaccctccgggcgtggtgggcgtggcccccactggcatcccagctgctgtggaaggtatcgtccccagcgccatgtccctgtcccacggcctgccccctgtggcccacccgccccacgccccttctcctggccaaaccgtcaagcccgaggcggatagagaccacgccagtgaccagttg

SEQ ID NO: 31 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 32 - DNA, 951 bp, Homo sapiens
  source 1..951: mol_type = unassigned DNA
atgtacaacatgatggagacggagctgaagccgccgggcccgcagcaaacttcggggggcggcggcggcaactccaccgcggcggcggccggcggcaaccagaaaaacagcccggaccgcgtcaagcggcccatgaatgccttcatggtgtggtcccgcgggcagcggcgcaagatggcccaggagaaccccaagatgcacaactcggagatcagcaagcgcctgggcgccgagtggaaacttttgtcggagacggagaagcggccgttcatcgacgaggctaagcggctgcgagcgctgcacatgaaggagcacccggattataaataccggccccggcggaaaaccaagacgctcatgaagaaggataagtacacgctgcccggcgggctgctggcccccggcggcaatagcatggcgagcggggtcggggtgggcgccggcctgggcgcgggcgtgaaccagcgcatggacagttacgcgcacatgaacggctggagcaacggcagctacagcatgatgcaggaccagctgggctacccgcagcacccgggcctcaatgcgcacggcgcagcgcagatgcagcccatgcaccgctacgacgtgagcgccctgcagtacaactccatgaccagctcgcagacctacatgaacggctcgcccacctacagcatgtcctactcgcagcagggcacccctggcatggctcttggctccatgggttcggtggtcaagtccgaggccagctccagcccccctgtggttacctcttcctcccactccagggcgccctgccaggccggggacctccgggacatgatcagcatgtatctccccggcgccgaggtgccggaacccgccgcccccagcagacttcacatgtcccagcactaccagagcggcccggtgcccggcacggccattaacggcacactgcccctctcacacatg

SEQ ID NO: 33 - protein, 264 aa, Homo sapiens
MLSHNTMMKQRKQQATAIMKEVHGNDVDGMDLGKKVSIPRDIMLEELSHLSNRGARLFKMRQRRSDKYTFENFQYQSRAQINHSIAMQNGKVDGSNLEGGSQQAPLTPPNTPDPRSPPNPDNIAPGYSGPLKEIPPEKFNTTAVPKYYQSPWEQAISNDPELLEALYPKLFKPEGKAELPDYRSFNRVATPFGGFEKASRMVKFKVPDFELLLLTDPRFMSFVNPLSGRRSFNRTPKGWISENIPIVITTEPTDDTTVPESEDL

SEQ ID NO: 34 - DNA, 792 bp, Homo sapiens
  source 1..792: mol_type = unassigned DNA
atgctatcacataatactatgatgaagcagagaaaacagcaagcaacagccatcatgaaggaagtccatggaaatgatgttgatggcatggacctgggcaaaaaggtcagcatccccagagacatcatgttggaagaattatcccatctcagtaaccgtggtgccaggctatttaagatgcgtcaaagaagatctgacaaatacacatttgaaaatttccagtatcaatctagagcacaaataaatcacagtattgctatgcagaatgggaaagtggatggaagtaacttggaaggtggttcgcagcaagcccccttgactcctcccaacaccccagatccacgaagccctccaaatccagacaacattgctccaggatattctggaccactgaaggaaattcctcctgaaaaattcaacaccacagctgtccctaagtactatcaatctccctgggaacaagccattagcaatgatccggagcttttagaggctttatatcctaaacttttcaagcctgaaggaaaggcagaactgcctgattacaggagctttaacagggttgccacaccatttggaggttttgaaaaagcatcaagaatggttaaatttaaagttccagattttgagctactattgctaacagatcccaggtttatgtcctttgtcaatcccctttctggcagacggtcctttaataggactcctaagggatggatatctgagaatattcctatagtgataacaaccgaacctacagatgataccactgtaccagaatcagaagaccta

SEQ ID NO: 35 - protein, 83 aa, Homo sapiens
MSFFQLLMKRKELIPLVVFMTVAAGGASSFAVYSLWKTDVILDRKKNPEPWETVDPTVPQKLITINQQWKPIEELQNVQRVTK

SEQ ID NO: 36 - DNA, 249 bp, Homo sapiens
  source 1..249: mol_type = unassigned DNA
atgagctttttccaactcctgatgaaaaggaaggaactcattcccttggtggtgttcatgactgtggcggcgggtggagcctcatctttcgctgtgtattctctttggaaaaccgatgtgatccttgatcgaaaaaaaaatccagaaccttgggaaactgtggaccctactgtacctcaaaagcttataacaatcaaccaacaatggaaacccattgaagagttgcaaaatgtccaaagggtgaccaaa

SEQ ID NO: 37 - protein, 653 aa, Homo sapiens
MAAAAASHLNLDALREVLECPICMESFTEEQLRPKLLHCGHTICRQCLEKLLASSINGVRCPFCSKITRITSLTQLTDNLTVLKIIDTAGLSEAVGLLMCRSCGRRLPRQFCRSCGLVLCEPCREADHQPPGHCTLPVKEAAEERRRDFGEKLTRLRELMGELQRRKAALEGVSKDLQARYKAVLQEYGHEERRVQDELARSRKFFTGSLAEVEKSNSQVVEEQSYLLNIAEVQAVSRCDYFLAKIKQADVALLEETADEEEPELTASLPRELTLQDVELLKVGHVGPLQIGQAVKKPRTVNVEDSWAMEATASAASTSVTFREMDMSPEEVVASPRASPAKQRGPEAASNIQQCLFLKKMGAKGSTPGMFNLPVSLYVTSQGEVLVADRGNYRIQVFTRKGFLKEIRRSPSGIDSFVLSFLGADLPNLTPLSVAMNCQGLIGVTDSYDNSLKVYTLDGHCVACHRSQLSKPWGITALPSGQFVVTDVEGGKLWCFTVDRGSGVVKYSCLCSAVRPKFVTCDAEGTVYFTQGLGLNLENRQNEHHLEGGFSIGSVGPDGQLGRQISHFFSENEDFRCIAGMCVDARGDLIVADSSRKEILHFPKGGGYSVLIREGLTCPVGIALTPKGQLLVLDCWDHCIKIYSYHLRRYSTP

SEQ ID NO: 38 - DNA, 1959 bp, Homo sapiens
  source 1..1959: mol_type = unassigned DNA
atggctgcagcagcagcttctcacctgaacctggatgccctccgggaagtgctagaatgccccatctgcatggagtccttcacagaagagcagctgcgtcccaagcttctgcactgtggccataccatctgccgccagtgcctggagaagctattggccagtagcatcaatggtgtccgctgtcccttttgcagcaagattacccgcataaccagcttgacccagctgacagacaatctgacagtgctaaagatcattgatacagctgggctcagcgaggctgtggggctgctcatgtgtcggtcctgtgggcggcgtctgccccggcaattctgccggagctgtggtttggtgttatgtgagccctgccgggaggcagaccatcagcctcctggccactgtacactccctgtcaaagaagcagctgaggagcggcgtcgggactttggagagaagttaactcgtctgcgggaacttatgggggagctgcagcggcggaaggcagccttggaaggtgtctccaaggaccttcaggcaaggtataaagcagttctccaggagtatgggcatgaggagcgcagggtccaggatgagctggctcgctctcggaagttcttcacaggctctttggctgaagttgagaagtccaatagtcaagtggtagaggagcagagttacctgcttaacattgcagaggtgcaggctgtgtctcgctgtgactacttcctggccaagatcaagcaggcagatgtagcactactggaggagacagctgatgaggaggagccagagctcactgccagcttgcctcgggagctcaccctgcaagatgtggagctccttaaggtaggtcatgttggccccctccaaattggacaagctgttaagaagccccggacagttaacgtggaagattcctgggccatggaggccacagcgtctgctgcctctacctctgttacttttagagagatggacatgagcccggaggaagtggttgccagccctagggcctcacctgctaaacagcggggtcctgaggcagcctccaatatccagcagtgcctctttctcaagaagatgggggccaaaggcagcactccaggaatgttcaatcttccagtcagtctctacgtgaccagtcaaggtgaagtactagtcgctgaccgtggtaactatcgtatacaagtctttacccgcaaaggctttttgaaggaaatccgccgcagccccagtggcattgatagctttgtgctaagcttccttggggcagatctacccaacctcactcctctctcagtggcaatgaactgccaggggctgattggtgtgactgacagctatgataactccctcaaggtatataccttggatggccactgcgtggcctgtcacaggagccagctgagcaaaccatggggtatcacagccttgccatctggccagtttgtagtaaccgatgtggaaggtggaaagctttggtgtttcacagttgatcgaggatcaggggtggtcaaatacagctgcctatgtagtgctgtgcggcccaaatttgtcacctgtgatgctgagggcaccgtctacttcacccagggcttaggcctcaatctggagaatcggcagaatgagcaccacctggagggtggcttttccattggctctgtaggccctgatgggcagctgggtcgccagattagccacttcttctcggagaatgaggatttccgctgcattgctggcatgtgtgtggatgctcgtggtgatctcatcgtggctgacagtagtcgcaaggaaattctccattttcctaagggtgggggctatagtgtccttattcgagagggacttacctgtccggtgggcatagccctaactcctaaggggcagctgctggtcttggactgttgggatcattgcatcaagatctacagctaccatctgagaagatattccacccca

SEQ ID NO: 39 - protein, 445 aa, Homo sapiens
MAEYDLTTRIAHFLDRHLVFPLLEFLSVKEIYNEKELLQGKLDLLSDTNMVDFAMDVYKNLYSDDIPHALREKRTTVVAQLKQLQAETEPIVKMFEDPETTRQMQSTRDGRMLFDYLADKHGFRQEYLDTLYRYAKFQYECGNYSGAAEYLYFFRVLVPATDRNALSSLWGKLASEILMQNWDAAMEDLTRLKETIDNNSVSSPLQSLQQRTWLIHWSLFVFFNHPKGRDNIIDLFLYQPQYLNAIQTMCPHILRYLTTAVITNKDVRKRRQVLKDLVKVIQQESYTYKDPITEFVECLYVNFDFDGAQKKLRECESVLVNDFFLVACLEDFIENARLFIFETFCRIHQCISINMLADKLNMTPEEAERWIVNLIRNARLDAKIDSKLGHVVMGNNAVSPYQQVIEKTKSLSFRSQMLAMNIEKKLNQNSRSEAPNWATQDSGFY

SEQ ID NO: 40 - DNA, 1335 bp, Homo sapiens
  source 1..1335: mol_type = unassigned DNA
atggcggagtacgacttgactactcgcatcgcgcactttttggatcggcatctagtctttccgcttcttgaatttctctctgtaaaggagatatataatgaaaaggaattattacaaggtaaattggaccttcttagtgataccaacatggtagactttgctatggatgtatacaaaaacctttattctgatgatattcctcatgctttgagagagaaaagaaccacagtggttgcacaactgaaacagcttcaggcagaaacagaaccaattgtgaagatgtttgaagatccagaaactacaaggcaaatgcagtcaaccagggatggtaggatgctctttgactacctggcggacaagcatggttttaggcaggaatatttagatacactctacagatatgcaaaattccagtacgaatgtgggaattactcaggagcagcagaatatctttatttttttagagtgctggttccagcaacagatagaaatgctttaagttcactctggggaaagctggcctctgaaatcttaatgcagaattgggatgcagccatggaagaccttacacggttaaaagagaccatagataataattctgtgagttctccacttcagtctcttcagcagagaacatggctcattcactggtctctgtttgttttcttcaatcaccccaaaggtcgcgataatattattgacctcttcctttatcagccacaatatcttaatgcaattcagacaatgtgtccacacattcttcgctatttgactacagcagtcataacaaacaaggatgttcgaaaacgtcggcaggttctaaaagatctagttaaagttattcaacaggagtcttacacatataaagacccaattacagaatttgttgaatgtttatatgttaactttgactttgatggggctcagaaaaagctgagggaatgtgaatcagtgcttgtgaatgacttcttcttggtggcttgtcttgaggatttcattgaaaatgcccgtctcttcatatttgagactttctgtcgcatccaccagtgtatcagcattaacatgttggcagataaattgaacatgactccagaagaagctgaaaggtggattgtaaatttgattagaaatgcaagactggatgccaagattgattctaaattaggtcatgtggttatgggtaacaatgcagtctcaccctatcagcaagtgattgaaaagaccaaaagcctttcctttagaagccagatgttggccatgaatattgagaagaaacttaatcagaatagcaggtcagaggctcctaactgggcaactcaagattctggcttctac

SEQ ID NO: 41 - protein, 156 aa, Homo sapiens
MKEPLDGECGKAVVPQQELLDKIKEEPDNAQEYGCVQQPKTQESKLKIGGVSSVNERPIAQQLNPGFQLSFASSGPSVLLPSVPAVAIKVFCSGCKKMLYKGQTAYHKTGSTQLFCSTRCITRHSSPACLPPPPKKTCTNCSKYKILNIPFYFTFF

SEQ ID NO: 42 - DNA, 468 bp, Homo sapiens
  source 1..468: mol_type = unassigned DNA
atgaaagaacctttggatggtgaatgtggcaaagcagtggtaccacagcaggagcttctggacaaaattaaagaagaaccagacaatgctcaagagtatggatgtgtccaacagccaaaaactcaagaaagtaaattgaaaattggtggtgtgtcttcagttaatgagagacctattgcccagcagttgaacccaggctttcagctttcttttgcatcatctggcccaagtgtgttgcttccttcagttccagctgttgctattaaggttttttgttctggttgtaaaaaaatgctttataagggccaaactgcatatcataagacaggatctactcagctcttctgctccacacgatgcatcaccagacattcttcacctgcctgcctgccacctcctcccaagaaaacctgcacaaactgctcgaagtataaaattcttaacatccctttttactttacctttttt

SEQ ID NO: 43 - protein, 155 aa, Homo sapiens
MQPSPPPTELVPSERAVVLLSCALSALGSGLLVATHALWPDLRSRARRLLLFLSLADLLSAASYFYGVLQNFAGPSWDCVLQGALSTFANTSSFFWTVAIALYLYLSIVRAARGPRTDRLLWAFHVVRWVAVALLFQEPPTQADPSRSCPPRGRV

SEQ ID NO: 44 - DNA, 465 bp, Homo sapiens
  source 1..465: mol_type = unassigned DNA
atgcagccgtccccgccgcccaccgagctggtgccgtcggagcgcgccgtggtgctgctgtcgtgcgcactctccgcgctcggctcgggcctgctggtggccacgcacgccctgtggcccgacctgcgcagccgggcacggcgcctgctgctcttcctgtcgctggccgacctgctctcggccgcctcctacttctacggagtgctgcagaacttcgcgggcccgtcgtgggactgcgtgctgcagggcgcgctgtccaccttcgccaacaccagctccttcttctggaccgtggccattgcgctctacttgtacctcagcatcgtccgcgccgcgcgcgggcctcgcacagatcgcctgctttgggccttccatgtcgtcaggtgggtggcggtggcgctgcttttccaggagcccccgacacaggccgacccctcccggtcttgccctcccagaggccgcgtc

SEQ ID NO: 45 - protein, 675 aa, Homo sapiens
MDTKSILEELLLKRSQQKKKMSPNNYKERLFVLTKTNLSYYEYDKMKRGSRKGSIEIKKIRCVEKVNLEEQTPVERQYPFQIVYKDGLLYVYASNEESRSQWLKALQKEIRGNPHLLVKYHSGFFVDGKFLCCQQSCKAAPGCTLWEAYANLHTAVNEEKHRVPTFPDRVLKIPRAVPVLKMDAPSSSTTLAQYDNESKKNYGSQPPSSSTSLAQYDSNSKKIYGSQPNFNMQYIPREDFPDWWQVRKLKSSSSSEDVASSNQKERNVNHTTSKISWEFPESSSSEEEENLDDYDWFAGNISRSQSEQLLRQKGKEGAFMVRNSSQVGMYTVSLFSKAVNDKKGTVKHYHVHTNAENKLYLAENYCFDSIPKLIHYHQHNSAGMITRLRHPVSTKANKVPDSVSLGNGIWELKREEITLLKELGSGQFGVVQLGKWKGQYDVAVKMIKEGSMSEDEFFQEAQTMMKLSHPKLVKFYGVCSKEYPIYIVTEYISNGCLLNYLRSHGKGLEPSQLLEMCYDVCEGMAFLESHQFIHRDLAARNCLVDRDLCVKVSDFGMTRYVLDDQYVSSVGTKFPVKWSAPEVFHYFKYSSKSDVWAFGILMWEVFSLGKQPYDLYDNSQVVLKVSQGHRLYRPHLASDTIYQIMYSCWHELPEKRPTFQQLLSSIEPLREKDKH

SEQ ID NO: 46 - DNA, 2025 bp, Homo sapiens
  source 1..2025: mol_type = unassigned DNA
atggatacaaaatctattctagaagaacttcttctcaaaagatcacagcaaaagaagaaaatgtcaccaaataattacaaagaacggctttttgttttgaccaaaacaaacctttcctactatgaatatgacaaaatgaaaaggggcagcagaaaaggatccattgaaattaagaaaatcagatgtgtggagaaagtaaatctcgaggagcagacgcctgtagagagacagtacccatttcagattgtctataaagatgggcttctctatgtctatgcatcaaatgaagagagccgaagtcagtggttgaaagcattacaaaaagagataaggggtaacccccacctgctggtcaagtaccatagtgggttcttcgtggacgggaagttcctgtgttgccagcagagctgtaaagcagccccaggatgtaccctctgggaagcatatgctaatctgcatactgcagtcaatgaagagaaacacagagttcccaccttcccagacagagtgctgaagatacctcgggcagttcctgttctcaaaatggatgcaccatcttcaagtaccactctagcccaatatgacaacgaatcaaagaaaaactatggctcccagccaccatcttcaagtaccagtctagcgcaatatgacagcaactcaaagaaaatctatggctcccagccaaacttcaacatgcagtatattccaagggaagacttccctgactggtggcaagtaagaaaactgaaaagtagcagcagcagtgaagatgttgcaagcagtaaccaaaaagaaagaaatgtgaatcacaccacctcaaagatttcatgggaattccctgagtcaagttcatctgaagaagaggaaaacctggatgattatgactggtttgctggtaacatctccagatcacaatctgaacagttactcagacaaaagggaaaagaaggagcatttatggttagaaattcgagccaagtgggaatgtacacagtgtccttatttagtaaggctgtgaatgataaaaaaggaactgtcaaacattaccacgtgcatacaaatgctgagaacaaattatacctggcagaaaactactgttttgattccattccaaagcttattcattatcatcaacacaattcagcaggcatgatcacacggctccgccaccctgtgtcaacaaaggccaacaaggtccccgactctgtgtccctgggaaatggaatctgggaactgaaaagagaagagattaccttgttgaaggagctgggaagtggccagtttggagtggtccagctgggcaagtggaaggggcagtatgatgttgctgttaagatgatcaaggagggctccatgtcagaagatgaattctttcaggaggcccagactatgatgaaactcagccatcccaagctggttaaattctatggagtgtgttcaaaggaataccccatatacatagtgactgaatatataagcaatggctgcttgctgaattacctgaggagtcacggaaaaggacttgaaccttcccagctcttagaaatgtgctacgatgtctgtgaaggcatggccttcttggagagtcaccaattcatacaccgggacttggctgctcgtaactgcttggtggacagagatctctgtgtgaaagtatctgactttggaatgacaaggtatgttcttgatgatcagtatgtcagttcagtcggaacaaagtttccagtcaagtggtcagctccagaggtgtttcattacttcaaatacagcagcaagtcagacgtatgggcatttgggatcctgatgtgggaggtgttcagcctggggaagcagccctatgacttgtatgacaactcccaggtggttctgaaggtctcccagggccacaggctttaccggccccacctggcatcggacaccatctaccagatcatgtacagctgctggcacgagcttccagaaaagcgtcccacatttcagcaactcctgtcttccattgaaccacttcgggaaaaagacaagcat

SEQ ID NO: 47 - protein, 502 aa, Homo sapiens
MASKKLGADFHGTFSYLDDVPFKTGDKFKTPAKVGLPIGFSLPDCLQVVREVQYDFSLEKKTIEWAEEIKKIEEAEREAECKIAEAEAKVNSKSGPEGDSKMSFSKTHSTATMPPPINPILASLQHNSILTPTRVSSSATKQKVLSPPHIKADFNLADFECEEDPFDNLELKTIDEKEELRNILVGTTGPIMAQLLDNNLPRGGSGSVLQDEEVLASLERATLDFKPLHKPNGFITLPQLGNCEKMSLSSKVSLPPIPAVSNIKSLSFPKLDSDDSNQKTAKLASTFHSTSCLRNGTFQNSLKPSTQSSASELNGHHTLGLSALNLDSGTEMPALTSSQMPSLSVLSVCTEESSPPNTGPTVTPPNFSVSQVPNMPSCPQAYSELQMLSPSERQCVETVVNMGYSYECVLRAMKKKGENIEQILDYLFAHGQLCEKGFDPLLVEEALEMHQCSEEKMMEFLQLMSKFKEMGFELKDIKEVLLLHNNDQDNALEDLMARAGAS

SEQ ID NO: 48 - DNA, 1506 bp, Homo sapiens
  source 1..1506: mol_type = unassigned DNA
atggcttctaagaagttgggtgcagattttcatgggactttcagttaccttgatgatgtcccatttaagacaggagacaaattcaaaacaccagctaaagttggtctacctattggcttctccttgcctgattgtttgcaggttgtcagagaagtacagtatgacttctctttggaaaagaaaaccattgagtgggctgaagagattaagaaaatcgaagaagccgagcgggaagcagagtgcaaaattgcggaagcagaagctaaagtgaattctaagagtggcccagagggcgatagcaaaatgagcttctccaagactcacagtacagccacaatgccacctcctattaaccccatcctcgccagcttgcagcacaacagcatcctcacaccaactcgggtcagcagtagtgccacgaaacagaaagttctcagcccacctcacataaaggcggatttcaatcttgctgactttgagtgtgaagaagacccatttgataatctggagttaaaaactattgatgagaaggaagagctgagaaatattctggtaggaaccactggacccattatggctcagttattggacaataacttgcccaggggaggctctgggtctgtgttacaggatgaggaggtcctggcatccttggaacgggcaaccctagatttcaagcctcttcataaacccaatggctttataaccttaccacagttgggcaactgtgaaaagatgtcactgtcttccaaagtgtccctcccccctatacctgcagtaagcaatatcaaatccctgtctttccccaaacttgactctgatgacagcaatcagaagacagccaagctggcgagcactttccatagcacatcctgcctccgcaatggcacgttccagaattccctaaagccttccacccaaagcagtgccagtgagctcaatgggcatcacactcttgggctttcagctttgaacttggacagtggcacagagatgccagccctgacatcctcccagatgccttccctctctgttttgtctgtgtgcacagaggaatcatcacctccaaatactggtcccacggtcacccctcctaatttctcagtgtcacaagtgcccaacatgcccagctgtccccaggcctattctgaactgcagatgctgtcccccagcgagcggcagtgtgtggagacggtggtcaacatgggctactcgtacgagtgtgtcctcagagccatgaagaagaaaggagagaatattgagcagattctcgactatctctttgcacatggacagctttgtgagaagggcttcgaccctcttttagtggaagaggctctggaaatgcaccagtgttcagaagaaaagatgatggagtttcttcagttaatgagcaaatttaaggagatgggctttgagctgaaagacattaaggaagttttgctattacacaacaatgaccaggacaatgctttggaagacctcatggctcgggcaggagccagc

SEQ ID NO: 49 - protein, 555 aa, Homo sapiens
MRVNGDDDSVAALSFLYDYYMGPKEKRILSSSTGGRNDQGKRYYHGMEYETDLTPLESPTHLMKFLTENVSGTPEYPDLLKKNNLMSLEGALPTPGKAAPLPAGPSKLEAGSVDSYLLPTTDMYDNGSLNSLFESIHGVPPTQRWQPDSTFKDDPQESMLFPDILKTSPEPPCPEDYPSLKSDFEYTLGSPKAIHIKSGESPMAYLNKGQFYPVTLRTPAGGKGLALSSNKVKSVVMVVFDNEKVPVEQLRFWKHWHSRQPTAKQRVIDVADCKENFNTVEHIEEVAYNALSFVWNVNEEAKVFIGVNCLSTDFSSQKGVKGVPLNLQIDTYDCGLGTERLVHRAVCQIKIFCDKGAERKMRDDERKQFRRKVKCPDSSNSGVKGCLLSGFRGNETTYLRPETDLETPPVLFIPNVHFSSLQRSGGAAPSAGPSSSNRLPLKRTCSPFTEEFEPLPSKQAKEGDLQRVLLYVRRETEEVFDALMLKTPDLKGLRNAISEKYGFPEENIYKVYKKCKRGILVNMDNNIIQHYSNHVAFLLDMGELDGKIQIILKEL

SEQ ID NO: 50 - DNA, 1665 bp, Homo sapiens
  source 1..1665: mol_type = unassigned DNA
atgagagtcaatggagatgatgacagtgttgcggccttgagcttcctctatgattactacatgggtcccaaggagaagcggatattgtcctccagcactgggggcaggaatgaccaaggaaagaggtactaccatggcatggaatatgagacggacctcactccccttgaaagccccacacacctcatgaaattcctgacagagaacgtgtctggaaccccagagtacccagatttgctcaagaagaataacctgatgagcttggagggggccttgcccacccctggcaaggcagctcccctccctgcaggccccagcaagctggaggccggctctgtggacagctacctgttacccaccactgatatgtatgataatggctccctcaactccttgtttgagagcattcatggggtgccgcccacacagcgctggcagccagacagcaccttcaaagatgacccacaggagtcgatgctcttcccagatatcctgaaaacctccccggaacccccatgtccagaggactaccccagcctcaaaagtgactttgaatacaccctgggctcccccaaagccatccacatcaagtcaggcgagtcacccatggcctacctcaacaaaggccagttctaccccgtcaccctgcggaccccagcaggtggcaaaggccttgccttgtcctccaacaaagtcaagagtgtggtgatggttgtcttcgacaatgagaaggtcccagtagagcagctgcgcttctggaagcactggcattcccggcaacccactgccaagcagcgggtcattgacgtggctgactgcaaagaaaacttcaacactgtggagcacattgaggaggtggcctataatgcactgtcctttgtgtggaacgtgaatgaagaggccaaggtgttcatcggcgtaaactgtctgagcacagacttttcctcacaaaagggggtgaagggtgtccccctgaacctgcagattgacacctatgactgtggcttgggcactgagcgcctggtacaccgtgctgtctgccagatcaagatcttctgtgacaagggagctgagaggaagatgcgcgatgacgagcggaagcagttccggaggaaggtcaagtgccctgactccagcaacagtggcgtcaagggctgcctgctgtcgggcttcaggggcaatgagacgacctaccttcggccagagactgacctggagacgccacccgtgctgttcatccccaatgtgcacttctccagcctgcagcgctctggaggggcagccccctcggcaggacccagcagctccaacaggctgcctctgaagcgtacctgctcgcccttcactgaggagtttgagcctctgccctccaagcaggccaaggaaggcgaccttcagagagttctgctgtatgtgcggagggagactgaggaggtgtttgacgcgctcatgttgaagaccccagacctgaaggggctgaggaatgcgatctctgagaagtatgggttccctgaagagaacatttacaaagtctacaagaaatgcaagcgaggaatcttagtcaacatggacaacaacatcattcagcattacagcaaccacgtcgccttcctgctggacatgggggagctggacggcaaaattcagatcatccttaaggagctg

SEQ ID NO: 51 - protein, 418 aa, Homo sapiens
MRSLLLLSAFCLLEAALAAEVKKPAAAAAPGTAEKLSPKAATLAERSAGLAFSLYQAMAKDQAVENILVSPVVVASSLGLVSLGGKATTASQAKAVLSAEQLRDEEVHAGLGELLRSLSNSTARNVTWKLGSRLYGPSSVSFADDFVRSSKQHYNCEHSKINFRDKRSALQSINEWAAQTTDGKLPEVTKDVERTDGALLVNAMFFKPHWDEKFHHKMVDNRGFMVTRSYTVGVMMMHRTGLYNYYDDEKEKLQIVEMPLAHKLSSLIILMPHHVEPLERLEKLLTKEQLKIWMGKMQKKAVAISLPKGVVEVTHDLQKHLAGLGLTEAIDKNKADLSRMSGKKDLYLASVFHATAFELDTDGNPFDQDIYGREELRSPKLFYADHPFIFLVRDTQSGSLLFIGRLVRPKGDKMRDEL

SEQ ID NO: 52 - DNA, 1254 bp, Homo sapiens
  source 1..1254: mol_type = unassigned DNA
atgcgctccctcctgcttctcagcgccttctgcctcctggaggcggccctggccgccgaggtgaagaaacctgcagccgcagcagctcctggcactgcggagaagttgagccccaaggcggccacgcttgccgagcgcagcgccggcctggccttcagcttgtaccaggccatggccaaggaccaggcagtggagaacatcctggtgtcacccgtggtggtggcctcgtcgctggggctcgtgtcgctgggcggcaaggcgaccacggcgtcgcaggccaaggcagtgctgagcgccgagcagctgcgcgacgaggaggtgcacgccggcctgggcgagctgctgcgctcactcagcaactccacggcgcgcaacgtgacctggaagctgggcagccgactgtacggacccagctcagtgagcttcgctgatgacttcgtgcgcagcagcaagcagcactacaactgcgagcactccaagatcaacttccgcgacaagcgcagcgcgctgcagtccatcaacgagtgggccgcgcagaccaccgacggcaagctgcccgaggtcaccaaggacgtggagcgcacggacggcgccctgttagtcaacgccatgttcttcaagccacactgggatgagaaattccaccacaagatggtggacaaccgtggcttcatggtgactcggtcctataccgtgggtgtcatgatgatgcaccggacaggcctctacaactactacgacgacgagaaggaaaagctgcaaatcgtggagatgcccctggcccacaagctctccagcctcatcatcctcatgccccatcacgtggagcctctcgagcgccttgaaaagctgctaaccaaagagcagctgaagatctggatggggaagatgcagaagaaggctgttgccatctccttgcccaagggtgtggtggaggtgacccatgacctgcagaaacacctggctgggctgggcctgactgaggccattgacaagaacaaggccgacttgtcacgcatgtcaggcaagaaggacctgtacctggccagcgtgttccacgccaccgcctttgagttggacacagatggcaacccctttgaccaggacatctacgggcgcgaggagctgcgcagccccaagctgttctacgccgaccaccccttcatcttcctagtgcgggacacccaaagcggctccctgctattcattgggcgcctggtccggcctaagggtgacaagatgcgagacgagtta

SEQ ID NO: 53 - protein, 636 aa, Homo sapiens
MSPHPEAITDCVTLNTVGQLAEGGYPLRFSTLFQEQQKMNISQASVSFKDVTIEFTQEEWQQMAPVQKNLYRDVMLENYSNLVSVGYCCFKPEVIFKLEQGEEPWFSEEEFSNQSHPKDYRGDDLIKQNKKIKDKHLEQAICINNKTLTTEEEKVLGKPFTLHVAAVASTKMSCKCNSWEVNLQSISEFIINNRNYSTKKIGCGNVCENSPFKINFEKTQTGEKFYEHNKNMKALNYNENLPKHPKFQTLEQAFECNKIGKAFNDKANCVKHNSSHTGETSSKDDEFRKNCDKKTLFDHRRTGTGKKHLHLNQCGKSFEKSTVEEYNKLNMGIKHYELNPSGNNFNRKAHLTDPQTAVIEENPLVSNDRTQTWVKSSEYHENKKSYQTSVHRVRRRSHSMMKPYKCNECGKSFCQKGHLIQHQRTHTGEKPFECSECGKTFSQKSHLSTHQRIHTAEKPYKCNECGKTFVQKSTLRGHQRIHTGEKPYECSECGKTFVQKSTLRDHHRIHTGEKSFQCNQCGKTFGQKSNLRIHQRTHTGEKTYQCNECEKSFWRKDHLIQHQKTHTGEKPFKCNECGKTFARTSTLRVHQRIHTGEKPFKCNECGKKFVRKAILSDHQRIHTGEKPFQCNKCGKT

SEQ ID NO: 54 - DNA, 1908 bp, Homo sapiens
  source 1..1908: mol_type = unassigned DNA
atgtcgccacatccagaagccatcacagattgtgtgacactgaacactgtgggccaacttgcagaaggtggttatcctttacggttctccacactctttcaggagcagcagaaaatgaacatatctcaggcatcagtgtcattcaaggacgtgactatagaattcacccaggaggagtggcagcaaatggcccctgttcagaagaatctgtacagagatgtgatgctggagaactacagcaacctcgtctcagtggggtactgctgtttcaaaccagaggtgatcttcaagttggagcaaggagaggagccttggttctcagaggaggaattctcaaaccagagtcacccaaaagattacagaggtgatgacctgatcaagcagaacaagaaaatcaaagacaaacacttggagcaagcaatatgtatcaataataaaacattgactacagaggaagagaaagttttggggaaaccatttactctgcatgtagctgctgttgcttcaacaaaaatgtcctgcaaatgcaactcatgggaagtgaatttgcaaagtatttctgaatttatcattaataatagaaactattcaacaaagaaaataggttgcggtaatgtatgtgagaattcacctttcaaaattaactttgagaaaactcagactggagagaaattttatgaacataataaaaacatgaaagctctcaattataatgaaaatcttcccaagcatccaaagtttcaaactttggagcaagcttttgaatgtaataaaattggaaaagcctttaatgataaggctaactgtgttaaacataacagttctcacacaggagaaacatcctctaaagatgatgaatttaggaaaaattgtgataagaaaactctctttgaccacaggagaactggcacagggaagaaacacctgcatcttaatcaatgtgggaaatcctttgagaagtcaactgtggaggaatataataaacttaatatgggtataaaacattatgaattaaatccaagtggaaataatttcaacagaaaggcacacctcactgatcctcaaacagctgtcatagaagagaacccattggtaagtaatgacagaacacagacttgggttaaatcctctgaatatcatgaaaataagaaatcctaccagacgtcggttcacagagttcgccgaagaagtcactcaatgatgaaaccctataaatgtaatgaatgtgggaaatccttctgtcagaaaggacatctcattcaacatcagagaactcacacaggagagaaaccatttgaatgtagtgaatgtggaaaaactttctcccagaagtcacacctcagtactcatcagagaattcatacagcagaaaaaccctataaatgtaatgaatgtggaaaaacatttgtccagaagtcaaccctcaggggacatcaaagaattcacacaggagaaaaaccctatgaatgtagtgaatgtgggaaaacttttgttcagaagtccaccctcagagatcatcacagaattcacacaggggagaaatcctttcaatgcaatcaatgtggaaaaacatttggccagaagtcaaacctcagaatacatcagagaactcacactggggagaaaacttaccagtgtaatgaatgtgaaaaatccttctggcgaaaagatcatctcattcaacatcagaaaactcacacgggagagaaaccattcaaatgtaacgaatgtgggaaaacttttgcccggacatcaaccctcagagtgcatcaaagaattcacactggggagaaaccatttaaatgtaacgaatgtgggaagaaatttgtccggaaagcaatccttagtgatcatcagagaattcacacaggggagaaaccctttcagtgtaataaatgtgggaaaact

SEQ ID NO: 55 - protein, 375 aa, Homo sapiens
MGCRRTREGPSKAMIFPWKCQSTQRDLWNIFKLWGWTMLCCDFLAHHGTDCWTYHYSEKPMNWQRARRFCRDNYTDLVAIQNKAEIEYLEKTLPFSRSYYWIGIRKIGGIWTWVGTNKSLTEEAENWGDGEPNNKKNKEDCVEIYIKRNKDAGKWNDDACHKLKAALCYTASCQPWSCSGHGECVEIINNYTCNCDVGYYGPQCQFVIQCEPLEAPELGTMDCTHPLGNFSFSSQCAFSCSEGTNLTGIEETTCGPFGNWSSPEPTCQVIQCEPLSAPDLGIMNCSHPLASFSFTSACTFICSEGTELIGKKKTICESSGIWSNPSPICQKLDKSFSMIKEGDYNPLFIPVAVMVTAFSGLAFIIWLARRLKKGM

SEQ ID NO: 56 - DNA, 1125 bp, Homo sapiens
  source 1..1125: mol_type = unassigned DNA
atgggctgcagaagaactagagaaggaccaagcaaagccatgatatttccatggaaatgtcagagcacccagagggacttatggaacatcttcaagttgtgggggtggacaatgctctgttgtgatttcctggcacatcatggaaccgactgctggacttaccattattctgaaaaacccatgaactggcaaagggctagaagattctgccgagacaattacacagatttagttgccatacaaaacaaggcggaaattgagtatctggagaagactctgcctttcagtcgttcttactactggataggaatccggaagataggaggaatatggacgtgggtgggaaccaacaaatctcttactgaagaagcagagaactggggagatggtgagcccaacaacaagaagaacaaggaggactgcgtggagatctatatcaagagaaacaaagatgcaggcaaatggaacgatgacgcctgccacaaactaaaggcagccctctgttacacagcttcttgccagccctggtcatgcagtggccatggagaatgtgtagaaatcatcaataattacacctgcaactgtgatgtggggtactatgggccccagtgtcagtttgtgattcagtgtgagcctttggaggccccagagctgggtaccatggactgtactcaccctttgggaaacttcagcttcagctcacagtgtgccttcagctgctctgaaggaacaaacttaactgggattgaagaaaccacctgtggaccatttggaaactggtcatctccagaaccaacctgtcaagtgattcagtgtgagcctctatcagcaccagatttggggatcatgaactgtagccatcccctggccagcttcagctttacctctgcatgtaccttcatctgctcagaaggaactgagttaattgggaagaagaaaaccatttgtgaatcatctggaatctggtcaaatcctagtccaatatgtcaaaaattggacaaaagtttctcaatgattaaggagggtgattataaccccctcttcattccagtggcagtcatggttactgcattctctgggttggcatttatcatttggctggcaaggagattaaaaaaaggtatg